(19) United States
(12) Patent Application Publication
(10) Pub. No.: US 2021/0386944 A1
(43) Pub. Date: Dec. 16, 2021

(54) GAS-EVOLVING COMPOSITIONS AND CONTAINER AND DELIVERY SYSTEMS

(71) Applicant: SaNOtize Research Development Corp., Vancouver (CA)

(72) Inventors: Dov Tamarkin, Tel Aviv (IL); Gilly Regev, Vancouver (CA); Christopher C. Miller, North Vancouver (CA)

(73) Assignee: SaNOtize Research Development Corp., Vancouver, BC (CA)

(21) Appl. No.: 17/284,465

(22) PCT Filed: Oct. 14, 2019
(86) PCT No.: PCT/IB2019/001103

§ 371 Date: Apr. 11, 2021

Related U.S. Application Data
(60) Provisional application No. 62/744,654, filed on Oct. 12, 2018.

(51) Int. Cl. A61M 15/00; A61M 11/04; A61K 45/06

(52) U.S. Cl. CPC A61M 15/009; A61M 15/0065; A61M 11/04; A61K 45/06; A61M 2202/04; A61M 2202/0233; A61M 2202/0275; A61M 2205/8218; A61M 2209/06; A61M 2202/0208

(57) ABSTRACT

A gas-evolving composition delivery system is disclosed, which comprises a pressurized container having a dispenser, and a liquid gas-evolving composition, such as an acidified nitrite solution, within the pressurized container, wherein the pressurized container has an internal pressure sufficient to minimize gas production from the gas-evolving composition while within the container. Also disclosed is a method of stabilizing an acidified nitrite solution comprising loading the solution into a container and sealing the container to be airtight.

### CROSS-REFERENCE TO RELATED APPLICATIONS

This application claims the benefit of U.S. Provisional Patent Application Ser. No. 62/744,654, filed on Oct. 12, 2018, which is incorporated herein by reference.

### BACKGROUND

Endogenous NO has plays a role in various bodily functions, including the vasodilatation of smooth muscle, neurotransmission, regulation of wound healing and immune responses to infection such as bactericidal action directed toward various organisms.

NO is a free-radical which is lipophilic with a small stokes radius making it a signaling molecule and enabling it to cross the plasma membrane into the cytosol. NO may play a role in wound healing through vasodilatation, angiogenesis, anti-inflammatory and antimicrobial action. When delivered in an exogenous gas form, the antimicrobial and cellular messenger regulatory properties of NO might easily enter the wound milieu and be useful in optimizing the healing of chronic wounds with specific actions directed at reducing bacterial burden, reducing exudate and improving endogenous debridement.

Further, the therapeutic potential of NO donors for cutaneous lesions, as a broad-spectrum antimicrobial may be promising. To date, this approach has not been realized in clinical commercial applications. This may be due to the toxic side effects of the carrier compounds of solid, liquid, cream, or other non-gaseous NO donors and specifically, the acidic environment for release of the NO molecule. Adequate efficacy also may not have been demonstrated due to binding of the nitric oxide with other compounds in the preparations. Endogenous approaches such as intracellular nitric oxide synthase (NOS) stimulation and exogenous wound dressings with either NO-donors or saturated NO-containing solutions have also failed to release consistent steady-state concentrations of NO.

Direct exposure to nitric oxide gas requires the patient to be connected to a gas cylinder for hours at a time for treatment. As such, these methods are not suitable or effective in situations when only a very short time is available for administration of the molecule. Thus, while NO and other gases can have a positive effect on biological systems, it can be impractical to apply such gases in their gas state to the skin, mucosal membranes, or body cavities in a way that can facilitate such biological effects or impart a therapeutic effect.

### BRIEF DESCRIPTION OF THE DRAWINGS

FIG. 1a illustrates an operation in the production of a gas-evolving kit in accordance with an example;

FIG. 1b illustrates an operation in the production of a gas-evolving kit in accordance with an example;

FIG. 1c illustrates an operation in the production of a gas-evolving kit in accordance with an example;

FIG. 1d illustrates an operation in the production of a gas-evolving kit in accordance with an example;

FIG. 1e illustrates an operation in the production of a gas-evolving kit in accordance with an example;

FIG. 2 illustrates a device comprising a canister, a metered-dose valve, and an actuator in accordance with an example;

FIG. 3 depicts the stability of an NO-evolving composition comprising sodium nitrate 40 millimolar (mM) and citric acid at a pH of 3.5 in accordance with an example;

FIG. 4a depicts a graph of concentration of a 20 millimolar (mM) nitric oxide releasing solution (NORS) with respect to time over a period of 24 hours in accordance with an example;

FIG. 4b depicts a graph of concentration of a 20 millimolar (mM) nitric oxide releasing solution (NORS) with respect to time over a period of 22 weeks in accordance with an example;

FIG. 4c depicts a graph of pH of a purged 20 millimolar (mM) nitric oxide releasing solution (NORS) with respect to time over a period of about 22 weeks in accordance with an example;

FIG. 4d depicts a graph of concentration of an unpurged 100 millimolar (mM) nitric oxide releasing solution (NORS) with respect to time over a period of 7 days in accordance with an example;

FIG. 4e depicts a graph of concentration of a purged 100 millimolar (mM) nitric oxide releasing solution (NORS) with respect to time over a period of 7 days in accordance with an example;

FIG. 4f depicts a graph of concentration of an unpurged 100 millimolar (mM) nitric oxide releasing solution (NORS) with respect to time over a period of 12 weeks in accordance with an example;

FIG. 4g depicts a graph of concentration of a purged 100 millimolar (mM) nitric oxide releasing solution (NORS) with respect to time over a period of 12 weeks in accordance with an example;

FIG. 4h depicts a graph of pH of a purged 100 millimolar (mM) nitric oxide releasing solution (NORS) and unpurged 100 mM NORS with respect to time over a period of about 90 days in accordance with an example;

FIG. 4i depicts a graph of concentration of 40 millimolar (mM) nitrogen with respect to time over a period of about 8 weeks in accordance with an example;

FIG. 4j depicts a graph of concentration of 40 millimolar (mM) nitrite with respect to time over a period of about 8 weeks in accordance with an example;

FIG. 4k depicts a graph of concentration nitrates from a 40 millimolar (mM) nitrite solution with respect to time over a period of about 8 weeks in accordance with an example;

FIG. 4l depicts a graph of pH of: purged 40 millimolar (mM) nitric oxide releasing solution (NORS), unpurged 40 millimolar (mM) nitric oxide releasing solution (NORS), and open 40 millimolar (mM) nitric oxide releasing solution (NORS) with respect to time over a period of about 8 weeks in accordance with an example;

FIG. 5a depicts a graph of nitric oxide (NO) production measured using a flow-over glass device and chemiluminescence in accordance with an example. 20 millimolar (mM) nitric oxide releasing solution (NORS) at pH 3.5 was left uncapped for 0, 10, 20, 30, 60, 120, 240 or 480 minutes before injection.

FIG. 5b depicts a graph of nitric oxide (NO) peak measured using the flow-over glass device and chemiluminescence in accordance with an example. 20 millimolar (mM) nitric oxide releasing solution (NORS) at pH 3.5 was left uncapped for 0, 10, 20, 30, 60, 120, 240 or 480 minutes before injection.

FIG. 5c depicts nitric oxide (NO) production two minutes after injection, measured using the flow-over glass device and chemiluminescence in accordance with an example. 20 millimolar (mM) nitric oxide releasing solution (NORS) at pH 3.5 was left uncapped for 0, 10, 20, 30, 60, 120, 240 or 480 minutes before injection.

FIG. 5d depicts the area under the curve after two minutes of measuring using the flow-over glass device and chemiluminescence in accordance with an example. 20 millimolar (mM) nitric oxide releasing solution (NORS) at pH 3.5 was left uncapped for 0, 10, 20, 30, 60, 120, 240 or 480 minutes before injection.

FIG. 6 depicts functionality of a gas-evolving composition delivery system in accordance with an example; and

FIG. 7 depicts a method of stabilizing an acidified nitrite solution in accordance with an example.

These drawings are provided to illustrate various aspects of the invention and are not intended to be limiting of the scope in terms of dimensions, materials, configurations, arrangements or proportions unless otherwise limited by the claims.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

The present invention relates to gas-evolving composition delivery systems and related methods. In one embodiment, the gas-evolving delivery system can include: a pressurized container having a dispenser, and a liquid gas-evolving composition within the pressurized container. The gas-evolving composition can comprise: a gas-evolving donor, an activator, and a pharmaceutically acceptable carrier. In one example, the pressurized container can have an internal pressure sufficient to minimize gas production of the gas-evolving composition while within the container.

The present invention also relates to methods of treating a disease or condition of a subject. In some aspects, the disease or condition can be a disease or condition that is responsive to nitric oxide therapy (e.g. which benefits from the administration or presence of nitric oxide). The method can include administering a gas-evolving composition to the subject from a pressurized container. The internal pressure of the pressurized container can minimize gas production of the gas-evolving composition prior to administration.

The present invention additionally relates to methods of controlling gas release from a gas-evolving composition. In one embodiment, the method can include subjecting a gas-evolving composition to a release-controlling pressure within a container to minimize gas release from the gas-evolving composition while in the container. In some embodiments, the gas-evolving composition can comprise: a gas-evolving donor, an activator, and a pharmaceutically acceptable carrier.

The present invention further relates to methods of stabilizing an acidified nitrite solution. In one aspect, such a method can include: loading the acidified nitrite solution into a container, and sealing the container to be airtight. In one embodiment, the acidified nitrite solution can comprise: a nitric oxide (NO) evolving donor; an NO activator; and a pharmaceutically acceptable carrier.

### Definitions

As used in this herein, the singular forms “a,” “an” and “the” include plural referents unless the context clearly dictates otherwise. Thus, for example, reference to “a cell” includes a plurality of such cells.

As used herein, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

As used herein, the terms “first,” “second,” “third,” “fourth,” and the like are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, the terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein.

As used herein, the term “coupled,” as used herein, is defined as directly or indirectly connected in a biological, chemical, mechanical, electrical or nonelectrical manner. “Directly coupled” structures or elements are in contact with one another and are attached. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the terms “therapeutic agent,” “active agent,” and the like can be used interchangeably and refer to an agent that can have a beneficial or positive effect on a subject when administered to the subject in an appropriate or effective amount.

As used herein, the term “modulate” is meant to refer to any change in biological state, i.e. increasing, decreasing, and the like.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent can be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel, it is recognized that individual variation and response to treatments may make the achievement of therapeutic effects a somewhat subjective decision. The determination of an effective amount or therapeutically effective amount is well within the ordinary skill in the art of pharmaceutical sciences and medicine.

As used herein, a “dosing regimen” or “regimen” such as “treatment dosing regimen,” or a “prophylactic dosing regimen” refers to how, when, how much, and for how long a dose of an active agent or composition can or should be administered to a subject in order to achieve an intended treatment or effect.

The term “treat” or “treatment,” and the like as used herein, refer to the alleviation (i.e., “diminution”) and/or the elimination of a sign or symptom or a source of a sign or symptom of a disease, disorder, or condition, either chronic or acute. Treatment can also be performed or administered for the purposes of prevention (e.g. prophylactic) of a disease, disorder, or condition and/or its signs or symptoms. By way of several non-limiting examples, a symptom of a bacterial infection can be treated by alleviating a symptom of that disorder. A symptom of a bacterial infection can also be treated by altogether eliminating a symptom of that disorder. A bacterial infection or colonization can be treated by alleviating the source, or “cause,” of that disorder. A bacterial infection or colonization can also be treated by eliminating the source of that disorder.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (e.g. solution), or gas. Furthermore, the term “dosage form” can include one or more formulation(s) or composition(s) provided in a format for administration to a subject. For example, an injectable dosage form would be a formulation or composition prepared in a manner that is suitable for administration via injection.

As used herein, a “subject” refers to an animal. In one aspect the animal may be a mammal. In another aspect, the mammal may be a human.

As used herein, the terms “treatment site,” “site of treatment,” “treatment situs,” and the like are used to mean an area, a region or a site on, or inside the body of, a subject, including a tissue, a wound, a cavity, an organ, a lesion, an abscess, including intact skin. The treatment sites that can be treated by the methods of the invention include any area, region or site on the surface of, or inside the body of, a subject that can be exposed to gaseous nitric oxide. By way of non-limiting examples, regions and sites that can be treated by the methods of the invention include, but are not limited to, external tissues (e.g. skin, etc.), internal tissues (e.g. mucosa, muscle, fascia, etc.), and internal organs (e.g. lungs, liver, etc.). It should be understood that many areas, regions and sites that are normally not amenable to exposure to gaseous nitric oxide can become amenable to exposure to gaseous nitric oxide after a wound, such as, for example, a surgical incision or traumatic laceration, is introduced to the body of a subject. Moreover, “treatment site” should not be construed to include only those areas, regions or sites that exhibit overt evidence of pathology, but rather should also be construed to include areas, regions or sites that may be asymptomatic, i.e., that do not contain overt evidence of pathology, but that may be affected nonetheless and that could, in time, exhibit more overt evidence of pathology. By way of non-limiting examples, such a site can include a trauma wound, surgical wound, intact tissue or burn, including those that have come into contact with, or which is at risk of potentially coming into contact with, a pathogen that can colonize or infect the wound, and can be treated, or prophylactically treated, with the devices and methods of the invention.

As used herein, “NORS” refers to a nitric oxide releasing solution or substance.

As used herein, “disease,” “disorder,” “condition,” and the like can be used interchangeably and refer to a physiologic abnormality either local or systemic which afflicts a subject. Diseases, disorders, and conditions can result from any number of causations such as pathogens, allergens, genetic or inherited anomalies, injury, environmental causes, etc.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, comparative terms such as “increasing,” “increased,” “decreasing,” “decreased,” “better,” “worse,” “higher,” “lower,” “enhancing,” “enhanced,” “maximizing,” “maximized,” “minimizing,” “minimized,” “ameliorating” and the like refer to a property, result, or effect of a device, composition, formula, component, treatment, regimen, method, or activity that is measurably different from a property, result, or effect of other devices, compositions, formulas, components, treatments, regimens, methods, or activities. Furthermore, comparative terms can refer to a different biological state, presence, absence, activity level, or operation that is measurably different than an endogenous biological state, presence, absence, activity level, or operation. Comparative terms can be used to indicate differences in a surrounding or adjacent area, for example, regions of tissue. Comparative terms can also be used to indicate differences in chemical or biological structure or activity (e.g. therapeutic activity or effectiveness). Additionally, comparative terms can be used to indicate differences in biologic or physiologic result, activity, or status as compared to a previous, or other biologic or physiologic result, activity, or status. For example, a process that has an “increased” therapeutic effect or result can refer to improved results or efficacy attained by the process as compared to a similar or different process intended for treatment of the same condition or experience. In the instance that a composition or treatment has been applied, such increase or decrease can be attributed to such composition or treatment. In other cases, the comparison can be made between the results achieved by two different applied formulations or treatment (e.g. formulations having different amounts of an active agent, etc.).

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 micrograms to about 80 micrograms” should also be understood to provide support for the range of “50 micrograms to 80 micrograms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

As mentioned previously, the present invention can relate to systems and methods for production and administration (e.g. topical administration) of a biologically-active gas for treatment using such systems and methods. The systems and methods can comprise a stable gas-evolving pharmaceutical or cosmetic composition, containing a gas-evolving donor, which, upon reaction with an activator, can yield a gas having therapeutic, medical, or cosmetic properties (e.g., a biologically-active gas). The composition can be housed in a pressurized container, having a positive pressure in it, that can have a dispenser. While the gas-evolving donor and the activator may be expected to react and evolve the corresponding biologically active gas, such a reaction can be inhibited by the pressure of the gas in the container.

The present invention can also relate to a method of stabilizing an acidified nitrite solution. The method can comprise loading the acidified nitrite solution into a container and sealing the container. The acidified nitrite solution can comprise a nitric oxide (NO) evolving donor, an NO activator, and a pharmaceutically acceptable carrier.

The present invention can also relate to a kit comprising a composition housing in a pressure-resistant container, having a positive pressure in it, equipped with a controllable outlet, such as a valve, equipped with a valve, comprising: a topically applicable vehicle; a gas-evolving composition; and an activator.

The present invention can also relate to a method of treating, alleviating, or preventing a dermatological, cosmetic, or mucosal disorder, or a disorder of a body cavity comprising administering topically to a subject having such a disorder a therapeutically effective amount of any of the compositions described herein.

### Packaging System

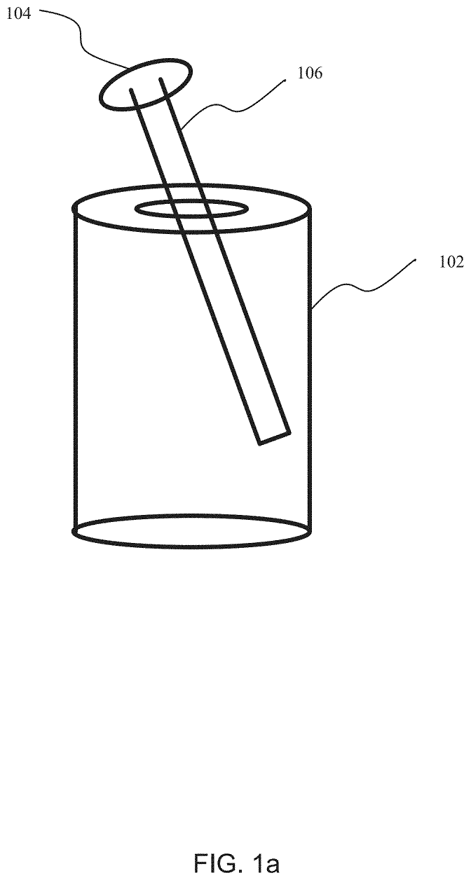

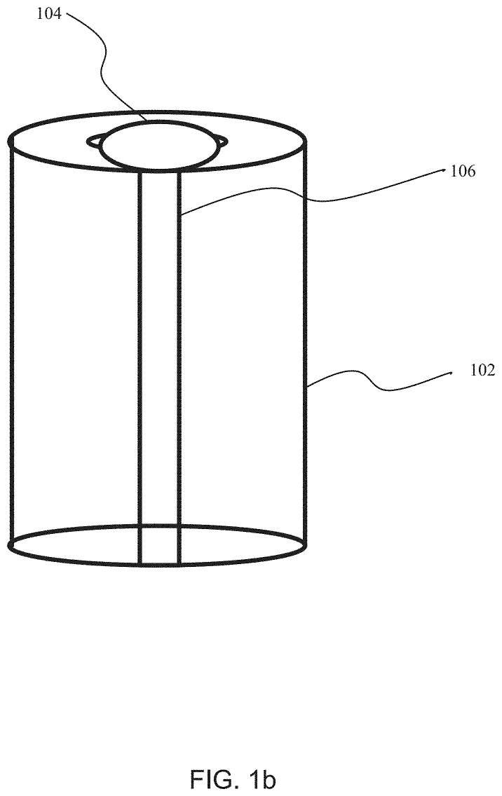

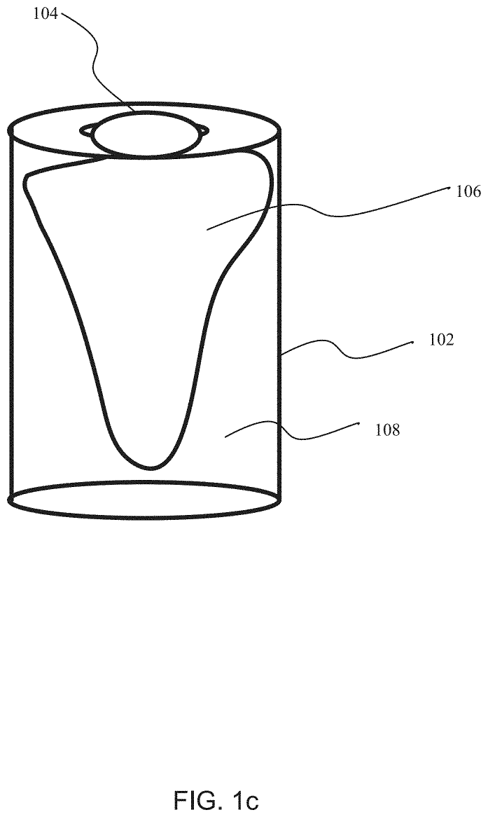

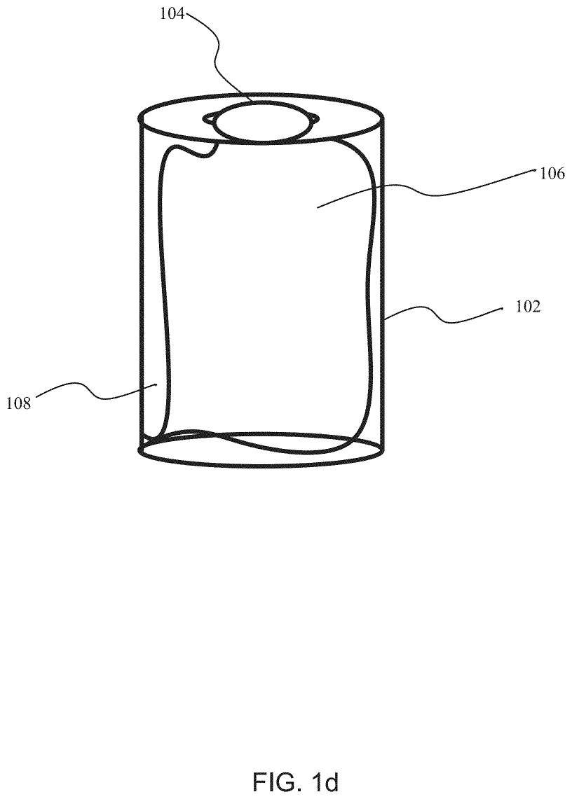

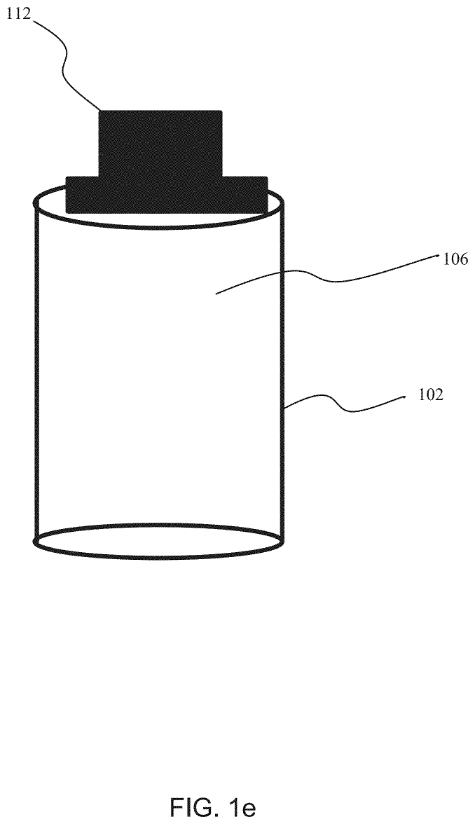

In one example, as illustrated in FIGS. 1a-1e, a gas-evolving kit can comprise: a gas-evolving composition, housed in a pressure-resistant container 102 which can be equipped with a controllable outlet that limits the production of gas. While thermodynamically the gas-evolving composition (e.g., a gas donor) and the activator can react and evolve the corresponding biologically active gas, such a reaction can be inhibited by the pressure of the gas in the container. The gas-evolving composition can further comprise a pharmaceutically acceptable carrier.

In an example, the pressure-resistant container 102 can be an aerosol canister, equipped with a valve 104. The container may be used in an upright position or in an up-side-down (inverted) position. The valve 104 may, or may not, be equipped with a tube 106, which can facilitate the flow of the composition to the valve 104. In one example, the internal pressure of the pressurized container can span a range between 150 kilopascals (kPa) to 1000 kPa.

In an example, the valve 104 affords, upon activation, a flow (e.g. a continuous flow) of the composition; yet in other examples, the valve 104 can be a metered dose valve, which, upon activation can dispense a set amount of composition, thus providing usage convenience and accuracy of the dose to be administered. In one or more examples, the metered dose valve 104 can provide a unit dose of between about 10 μL and about 1000 μL, while in other examples, the dose can be more than about 1000 μL.

In an example, a propellant can be used to generate a positive pressure inside the gas-resistant container 102. The positive pressure can be useful to inhibit the production of the biologically-active drug; and to facilitate dispensing the composition from the container 102.

In another example, pressure within the container that is sufficient to dispense the composition can be created by an external force, such as by a subject squeezing the container.

In an example, the production of the kit can comprise the operations: (1) fill the can 102 with the gas-evolving composition, comprising a vehicle, the gas donor and the activator; (2) crimp the can 102 with a valve 104, (3) add a propellant into the can 102 and the bag 106 (during crimping or after crimping); (4) assemble an actuator 112, suitable for dispensing the gas-evolving composition. In both cases, pressure can be implied.

In an example, the pressure-resistant container 102 can be a “bag on valve”, a “bag-in-can”, a “piston can” or a “can-in-can” aerosol container, in which a bag 106 can house the gas-evolving composition and can be attached directly to the valve 104 or an internal can that can be inserted inside an external can 102.

In an example, the operations of production of a bag on valve, or can in can kit can comprise: (1) insert the bag 106 on valve 104 assembly, or the internal can, into the external can 102, as illustrated in FIG. 1a; (2) crimp the valve 104 onto the external can 102, as illustrated in FIG. 1b; (3) fill the bag 106, or internal can, with the gas-evolving composition, comprising a vehicle, the gas donor and the activator, as illustrated in FIG. 1c; (4) add a propellant into the space 108 between the can and the bag, or internal can (during crimping or following crimping), as illustrated in FIG. 1d; (5) assemble an actuator 112 suitable for dispensing the gas-evolving composition, as illustrated in FIG. 1e. FIGS. 1a-1e illustrates this sequence in an exemplary way for a bag in valve system.

In an example, the operations for production of a “bag-in-can”, a “piston can,” or a “can-in-can” aerosol containers can vary based on their different mechanical structures, but the principles of assembling the system, filling it with the gas-evolving composition, and closing it can have similarities to the operations described in the preceding.

In an example, the pressure-resistant container can be an inhaler device for respiratory therapy, which can facilitate treatment of organs of the respiratory system including: mucosal membranes, the oral cavity, the nasal cavity, the sinuses, the pharnix, the larynx, the trachea, the bronchus, and the lungs.

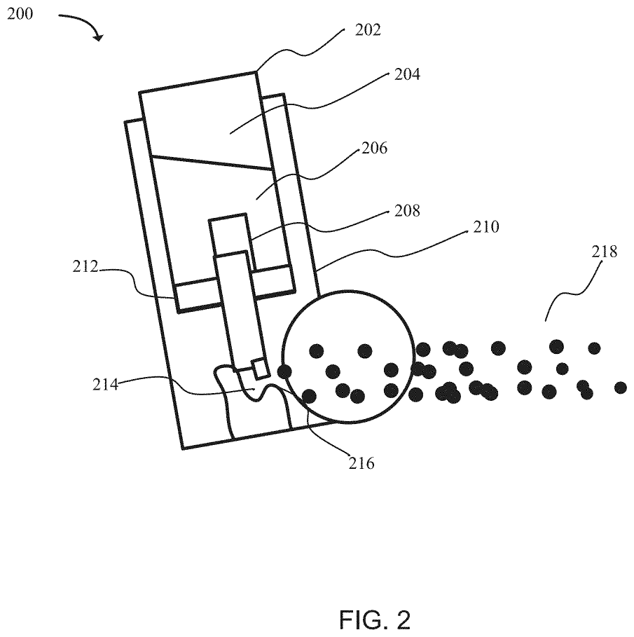

In another example, as illustrated in FIG. 2, a device 200 can comprise a canister 202, a metered-dose valve 212, and an actuator 210 that enables the release of a spray 218. The process of using such a device 200 can include: (a) when pressing the canister 202 into the actuator 210, the gas-evolving composition 206 or propellant 204 mixture in the metering valve 212 can be released under pressure from a retaining cup 208; (b) as the propellant 204 ejects from the pressurized valve 212 into ambient pressure in an expansion chamber 214 through the actuator nozzle 216, it can “flash” (i.e., rapidly expand and vaporize). This expansion and vaporization can shear and break the liquid stream into an aerosol; (c) propellant vaporization can result in cooling of the liquid-gas aerosol suspension; (d) upon release, the metering valve 212 can re-fill with the mixture of gas-evolving composition 206 and propellant 204 from the bulk of the canister 202, and be ready for the next discharge.

In an example, the pressure-resistant container can be coated with an inert lacquer, to prevent chemical interactions between the said container and the gas-evolving composition. Examples of inert lacquers can include epoxy polymers, such as phenol epoxy, vinyl polymers, rubber, polyamide-imide (PAM), acrylic polymers and polytetrafluoroethylene.

### Biologically-Active Gases

In one example, the composition can comprise at least one gas donor and at least one activator which, upon dispensing from the packaging system, can evolve a biologically-active gas, which can provide a therapeutic or cosmetic activity. The biologically-active gases useful herein can in some instances provide more than one benefit or operate via more than one mode of action. Therefore, classifications herein are made for the sake of convenience and are not intended to limit the active agent to that particular application or applications listed.

### NO Releasing Solutions

In one example, nitric oxide, having the molecular formula NO, upon release from a composition, can migrate fast to reach its target site. The chemical properties of NO can include its function as a transcellular signal in the cardiovascular and nervous systems and as a cytotoxic antipathogenic agent released during an inflammatory response. Among other properties, NO can be an antibiotic. The term “antibiotic” as used herein can include, but is not limited to, a destructive or inhibitory effect on the growth of bacteria; or the capacity to inhibit the growth of or to destroy bacteria. NO can be effective in eradicating fungi, yeast, molds and viruses. NO can further have anti-inflammatory effects, skin revitalizing effects, wound healing effects, and be used to treat various dermatoses and keratoses. Upon penetration into and through the skin and other tissues, NO can cause peripheral vasodilation, thereby lowering systolic and diastolic blood pressure and increasing dermal blood flow.

In one example, the NORS disclosed herein can provide an immediate or an extended release of gNO to a subject in need thereof. By “extended release,” it is meant that an effective amount of NO gas can be released from the formulation at a controlled rate such that therapeutically beneficial levels (but below toxic levels) of the component can be maintained over an extended period of time ranging from, e.g., about 5 seconds to about 24 hours, thus providing a 30 to 60 minute, or several hours, dosage form.

In one example, the NO gas can be released over a period of at least 30 minutes. In another embodiment, the NO gas can be released over a period of at least 8 hours. In another embodiment, the NO gas can be released over a period of at least 12 hours. In another embodiment, the NO gas can be released over a period of at least 24 hours. An extended release NORS can be beneficial in that the solution can be administered to the subject over a short period of time, while the release of NO from the solution can continue following administration. Moreover, the use of an extended release NORS can allow the subject to remain ambulatory following administration of the solution, as opposed to remaining stationary while being connected to a NO-releasing device in order to receive treatment.

In another example, the solution can become active when the nitrites and acids mix in saline or water in which the pH of the solution is below 4.0 and can exhibit an increased or enhanced production level of nitric oxide gas over an extended period of time. In one example, the pH of the active state of the nitric oxide releasing solution can be between a pH of about 1.0 and a pH of about 6.0. In another example, the pH of the active state of the nitric oxide releasing solution can be between a pH of about 3.0 and a pH of about 4.0. In one example, the pH can be about 3.2. In another example, the pH can be about 3.6. In another example, the pH can be about 3.7. In one example, the pH can be about 4.0. In another example, the pH can be below about 4.0.

In one example, the pH of the solution can be lowered via addition of at least one acidifying agent into the solution. Introduction of the acidifying agent can drive the solution reaction towards the reactants, thus reducing the pH (creating more acid), which in turn can create more nitric oxide gas.

For example, by introducing sodium nitrite (or other salts of nitrites) to a saline solution it can very slowly produce nitric oxide gas, but in an undetectable amount (as measured by chemiluminescence analysis methodology (ppb sensitivity)). The rate of NO produced from the solution can increase as the pH decreases, particularly as it drops below pH 4.0. NO can be produced based on the following equilibrium equations:

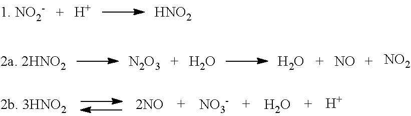

Therefore, an acidifying agent, for example an acid, may donate the H⁺ to the nitrite (NO₂⁻). The more H⁺ present, the faster the reaction can go towards HNO₂ and the more NO can be produced.

In one example, the nitric oxide releasing solution can include the use of a water- or saline-based solution and at least one nitric oxide releasing compound, such as nitrite or a salt thereof. In one example, the solution can be a saline-based solution. In one example, the nitric oxide releasing compound can be a nitrite, a salt thereof, and any combinations thereof. Non-limiting examples of nitrites can include salts of nitrite such as sodium nitrite, potassium nitrite, barium nitrite, and calcium nitrite, mixed salts of nitrite such as nitrite orotate, and nitrite esters such as amyl nitrite. In one example, the nitric oxide releasing compound can be selected from the group consisting of sodium nitrite and potassium nitrite, and any combinations thereof. In another example, the nitric oxide releasing compound can be sodium nitrite. In one example, the solution can be comprised of sodium nitrite in a saline solution. In another example, the solution can be comprised of potassium nitrite in a saline solution.

In another example, the NO donor can be selected from several classes, including, but not limited to inorganic nitrite and nitrate salts (e.g., sodium nitrite and sodium nitrate), organic nitrites and nitrates, sodium nitroprusside, molsidomine and its metabolites, diazeniumdiolates, S-nitrosothiols, mesoionic oxatriazole and derivatives thereof, iron-sulphur nitrosyls, Sinitrodil, and derivatives thereof.

In one example, an organic NO donor can include at least one organic nitrite, which can include esters of nitric acid and may be an acyclic or cyclic compound. For instance, the organic nitrate can be ethylene glycol dinitrate; isopropyl nitrate; amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, nitroglycerin, butane-1,2,4-triol-trinitrate; erythrityl tetranitrate; pentaerythrityl tetranitrate; sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatylnitrate, nitrite esters of sugars, nitrate esters of sugars, nitrite esters of polyols, nitrate esters of polyols, nicorandil, apresoline, diazoxide, hydralazine, hydrochlorothiazide, minoxidil, pentaerythritol, tolazoline, scoparone (6,7-dimethoxycoumarin) and pharmaceutically acceptable salts, isomers, anlogs, or derivatives thereof.

In one example, the concentration of nitrites in the solution can be from about 0.07% w/v to about 4% w/v. In one example, the concentration of nitrites in the solution can be no greater than about 0.5, 1, 2, or 3% w/v. In another example, the concentration of nitrites in the solution can be between about 0.07-1% w/v. In some embodiments, the concentration of nitrites can be an amount that is selected in order to compensate for any anticipated nitric oxide production (i.e. nitrite loss) during storage based on a selected container in which the composition will be placed. For example, if the container has properties (e.g. shape that dictates headspace amount, particular dispensing or other internal structure, type of seal, flexibility of material, etc.) that will cause an anticipated production of nitric oxide that will reduce the concentration of nitrites in solution by 1% w/v, then a higher concentration of nitrites may be added to the solution which anticipate the reduction during storage and thus compensate for the loss and allow the composition to provide a correct anticipated dosage when administered to a subject. As used herein, the term “w/v” can refer to the (weight of solute(gr)/volume of solution(ml))×100%.

In another example, the composition may also include at least one acidifying agent. As discussed previously, the addition of at least one acidifying agent to the solution can contribute towards increased production of nitric oxide. In one example, the acidifying agent can be an acid. Non-limiting examples of acids include ascorbic acid, ascorbyl palmitate, salicylic acid, malic acid, lactic acid, citric acid, formic acid, benzoic acid, tartaric acid, hydrochloric acid, sulfuric acid, and phosphoric acid. In one example, the acid can be selected from the group consisting of ascorbic acid, citric acid, malic acid, hydrochloric acid, and sulfuric acid, and any combinations thereof. In one example, the acid can be citric acid.

In another example, the amount of acidifying agent in the solution can directly affect the rate of the reaction to produce nitric oxide. In one example, the amount of acidifying agent from about 0.07% w/v to about 4% w/v. In another example, the amount of acidifying agent can be about 0.5, 1, 2, or 3% w/v. In another example, the amount of acidifying agent can be about 0.2% w/v. In one example, the amount of acidifying agent can be about 0.07% w/v. In another example, the amount of acidifying agent can be between about 0.07-2% w/v. Additionally, as recited above with respect to the nitrites concentration in the solution, the acidifying agent can also be included in an amount that accounts for or otherwise anticipates an amount of nitric oxide production within the container during storage, given the container's particular properties and/or characteristics. Such amounts of nitrites and/or acidifying agents and/or compositions containing such can be referred to as “container-compensated” compositions, elements, constituents, ingredients, or the like, and can also be referred to “storage-fortified” compositions, elements, constituents, ingredients, or the like. As such, a “container-compensated amount” or a “storage-fortified amount” of either a nitrite component or an acidifying agent would be an amount of the component that is calculated to compensate for any loss during storage due to nitric oxide production within the container and still deliver a target dosage or dosage within a target range to a subject upon administration to the subject following storage.

In one example, the activator can be a therapeutically-active acid, i.e., an acid that can have a benefit when applied to a tissue such as the skin. Examples of therapeutically-active acids can include, but are not limited to alpha-hydroxy acids, beta-hydroxy acids, citric acid, ascorbic acid, lactic acid, glycolic acid and salycilic acid. Additional suitable hydroxy acids include but are not limited to agaricic acid, aleuritic acid, allaric acid, altraric acid, arabiraric acid, ascorbic acid, atrolactic acid, benzilic acid, citramalic acid, dihydroxytartaric acid, erythraric acid, galactaric acid, galacturonic acid, glucaric acid, glucuronic acid, glyceric acid, gularic acid, gulonic acid, hydroxypyruvic acid, idaric acid, isocitric acid, lyxaric acid, malic acid, mandelic acid, mannaric acid, methyllactic acid, mucic acid, phenyllactic acid, pyruvic acid, quinic acid, ribaric acid, ribonic acid, saccharic acid, talaric acid, tartaric acid, tartronic acid, threaric acid, tropic acid, uronic acids and xylaric acid. Most of the above mentioned therapeutically-active acids can be weak acids that can be used as activators on their own or in a buffer system combination.

In one example, the stoichiometric (molar) amount of the NO donor can be less than the activator (so that there are more moles of activator than moles of NO donor). In one example, the effect of the combination of NO and the therapeutically-active activator can be synergistic.

In one example, nitric oxide can be generated by reduction of nitric acid with copper: 8 HNO3+3 Cu→3 Cu(NO3)2+4 H2O+2 NO. In this example, the NO donor can be nitric acid and the activator can be copper. Additional activators can include other metals, such as iron, magnesium, and the like.

In one example, each of the following reactions can also produce NO. These reactions can involve the reduction of nitrous acid in the form of sodium nitrite or potassium nitrite:

2NaNO2+2NaI+2H2SO4→I2+4NaHSO4+2NO

2NaNO2+2FeSO4+3H2SO4→Fe2(SO4)3+2NaHSO4+2H2O+2NO

3KNO2+KNO3+Cr2O3→2K2CrO4+4NO

The NO donor in these three examples can be a salt of nitrous acid and the activator can be sodium iodide and an acid, iron sulfate and an acid; and potassium nitrate and a chromate salt.

In one example, additional NO donors can include NONOate compounds, having the chemical formula R1R2N—(NO—)—N═O, where R1 and R2 are alkyl groups. One example is 1,1-diethyl-2-hydroxy-2-nitrosohydrazine, or diethylamine dinitric oxide. These compounds can have three sequential nitrogen atoms: an amine functional group, a bridging NO— group, and a terminal nitrosyl group. In contact with water, these compounds can release NO and can also be used for nitric oxide generation. This process can be pH-dependent and can be accelerated at lower pH values. In this example, the activator can be a pH-controlling agent, such as an acid or a buffer system.

In one example, the stoichiometric (molar) amount of the NO donor can be less than the activator (so that there are more moles of activator than moles of NO donor). In one example, the effect of the combination of NO and the therapeutically-active activator can be synergistic.

In one example, the NO donor can be linked to a polymer. In one example, the NO donor can be N-diazeniumdiolate. In one example, the NO donor can comprise N-diazeniumdiolate bound to a polymer; and in an additional example, the NO donor can comprise a polysiloxane polymer backbone that can contain covalently bound N-diazeniumdiolate nitric oxide donors throughout the polymeric structure, as provided in the following:

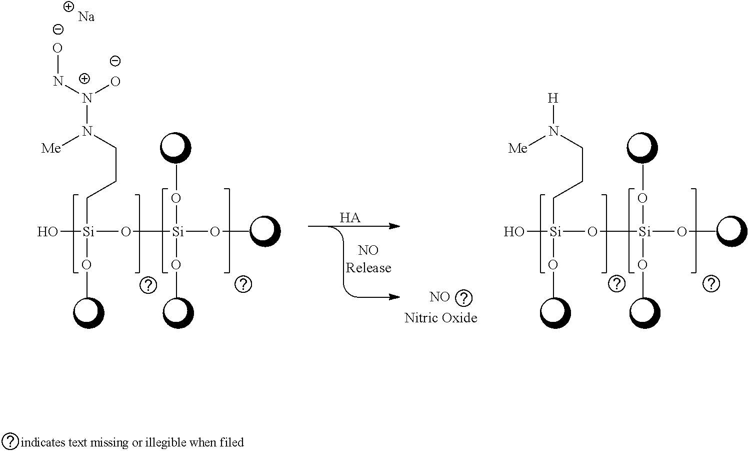

In one example, the NO donor can be bound to a polymer and the activator can be an acid. In one example, the NO donor can be a nitrite salt (MN02, wherein M is a cationic metal), such as sodium nitrite; and the activator can be an acid (HA). The acid can be an inorganic acid (such as hydrochloric acid nitric acid, sulfuric acid and phosphoric acid or an organic acid).

In one example, the acid activator can be a therapeutically-active acid, i.e., an acid that can have a benefit when applied to a tissue, such as the skin. Examples of therapeutically-active acids can include, but are not limited to alpha-hydroxy acids, beta-hydroxy acids, citric acid, ascorbic acid, lactic acid, glycolic acid and salicylic acid. Additional suitable hydroxy acids include but are not limited to agaricic acid, aleuritic acid, allaric acid, altraric acid, arabiraric acid, ascorbic acid, atrolactic acid, benzylic acid, citramalic acid, dihydroxytartaric acid, erythraric acid, galactaric acid, galacturonic acid, glucaric acid, glucuronic acid, glyceric acid, gularic acid, gulonic acid, hydroxypyruvic acid, idaric acid, isocitric acid, lyxaric acid, malic acid, mandelic acid, mannaric acid, methyllactic acid, mucic acid, phenyllactic acid, pyruvic acid, quinic acid, ribaric acid, ribonic acid, saccharic acid, talaric acid, tartaric acid, tartronic acid, threaric acid, tropic acid, uronic acids and xylaric acid.

In one example, any other compounds that can release NO upon reaction with an activator can be suitable as a NO donor. In one example, the pH can be selected to provide a gas pressure below the maximum pressure that can be sustained by the pressure-resistant container. In one example, the effect of the combination of nitric oxide and the therapeutically-active activator can be synergistic.

In another example, the solution can release a therapeutically effective concentration of NO. In one example, the therapeutically effective concentration of NO can be between about 100 ppm and about 1000 ppm. In another example, the therapeutically effective concentration of NO can be between about 120 ppm and about 400 ppm. In another example, the therapeutically effective concentration of NO can be about 160-250 ppm. Such concentrations may be measured within a specific window of time, or for a defined, or pre-defined duration. Such concentrations can be measured in terms of total release within the time period, or in terms of average release per hour for the time period. For example, in one embodiment, the concentration of NO released can be at least about 2 ppm for a time period of 24 hours or an average of 2 ppm/hour for 24 hours. In another embodiment, the concentration of NO can be at least about 20 ppm for a period of 12 hours, or an average of 20 ppm/hour for a period of 12 hours. In one embodiment, the concentration of NO released from the solution can be from an average of about 1 ppm/hr for a time period of 12 hours to an average of about 20 ppm/hr for about 24 hours.

### Carbon Dioxide

In one example, carbon dioxide, having the molecular formula CO2, is a very small molecule that, upon release from a composition, can migrate fast and reach its target site. It can afford multiple potential biological activities. For example, CO2 can treat peripheral edema in cancer patients receiving cancer treatment such as surgery or radiotherapy. (www.jpsmjournal.com/article/S0885-3924(14)00401-1/pdf). CO2 can be used to treat acid burns and their long-term symptoms, e.g., skin ulceration and severe pain. (www.ncbi.nlm.nih.gov/pubmed/2236319). CO2 can be added to local anesthetics, to speed up the onset of their effects and make their injection less painful. (www.frca.co.uk/article.aspx?articleid=100505).

In one example, while a precursor of CO2 (e.g., sodium carbonate or other salts of carbonate ion in solution) can be used, the release of CO2 directly to the target site can be of benefit. Various compounds can be CO2 donors. In one example, the CO2 donor can be a carbonate. The carbonate can be a salt of at least one metal ion or hydrogen ion, and carbonate (MCO3), or any other carbonate salt, compound, conjugate or complex. By way of example, the following equation shows the equilibrium reaction of a carbonate salt and an acid: MCO3+HA [symbol] MC1+H2O+CO2, wherein M is at least one metal ion or hydrogen ion and A is an acid. In one example, any compound capable of releasing CO2 upon reaction with an activator can be a CO2 donor.

In one example, the activator for CO2 can be an acid. In certain examples, the acid activator can be a therapeutically-active acid (i.e., an acid that has a benefit when applied to a tissue, such as the skin). Examples of therapeutically-active acids can include, but are not limited to alpha-hydroxy acids, beta-hydroxy acids, citric acid, ascorbic acid, lactic acid, glycolic acid and salicylic acid. Additional suitable hydroxy acids can include but are not limited to agaricic acid, aleuritic acid, allaric acid, altraric acid, arabiraric acid, ascorbic acid, atrolactic acid, benzylic acid, citramalic acid, dihydroxytartaric acid, erythraric acid, galactaric acid, galacturonic acid, glucaric acid, glucuronic acid, glyceric acid, gularic acid, gulonic acid, hydroxypyruvic acid, idaric acid, isocitric acid, lyxaric acid, malic acid, mandelic acid, mannaric acid, methyllactic acid, mucic acid, phenyllactic acid, pyruvic acid, quinic acid, ribaric acid, ribonic acid, saccharic acid, talaric acid, tartaric acid, tartronic acid, threaric acid, tropic acid, uronic acids and xylaric acid.

In one example, the amount of the concentration of the acid can be selected to achieve a specific pH range, which can provide a reaction of the acid and the gas donor in a specific rate. The pH of the composition can be between 1 and 7; or between 1 and 2 or between 2 and 3; or between 3 and 4 or between 5 and 6 or between 6 and 7.

In one example, the desirable pH range can be achieved by preparing a buffer solution (which can serve as an activator). The buffer can comprise, for example a weak acid and a salt of a weak acid; or a weak base and a salt of a weak base; or a mixture of a weak acid and a weak base. The ratio between the buffer components can determine the pH range. In one example, the ratio between the buffer components can be selected to obtain a pH range of between 1 and 7; or between 1 and 2 or between 2 and 3; or between 3 and 4 or between 5 and 6 or between 6 and 7.

Another consideration for determining the pH of the composition can be its compatibility with the organ to be treated. For example, if the target application site is the skin, the pH range can be between 3 and 7. This consideration can be applied to any other target site, such as mucosal membranes, the anum, the rectum, the GI system, the vagina, the penile urethra, the eye, the respiratory system, including the oral cavity, the nasal cavity, the sinuses, the pharnix, the larynx, the trachea, the bronchus and the lungs, the dental system, and the ear canal. In one example, the pH of the composition can be compatible with the skin, mucosal membranes, the anum, the rectum, the GI system, the vagina, the penile urethra, the eye, the respiratory system, including the oral cavity, the nasal cavity, the sinuses, the pharnix, the larynx, the trachea, the bronchus and the lungs, the dental system, and the ear canal.

In one example, the pH can be selected to provide a gas pressure which can be below the maximum pressure that can be sustained by the pressure-resistant container. In some examples, the effect of the combination of CO2 and the therapeutically-active activator can be synergistic.

### Oxygen

In one example, oxygen, having the molecular formula O2, can be a very small molecule that, upon release from a composition, can migrate fast and reach its target site. Oxygen can have multiple potential biological activities. For example, as an oxidizing agent, O2 can be an antibiotic. The term “antibiotic” as used herein includes, but is not limited to, a destructive or inhibitory effect on the growth of bacteria; or the capacity to inhibit the growth of or to destroy bacteria. Likewise, O2 can be effective against fungi, yeast, molds and viruses. O2 can further have skin-revitalizing effects by providing more oxygen to the living tissue. O2 can further be used to treat various dermatoses and keratoses. O2 can encourage wound healing.

In one example, while precursors of O2 in solution, such as H2O2 and hypochlorite compounds can be used topically, the release of O2 directly to the target site may be of benefit. Various compounds can be O2 donors. In one example, the O2 donor can be hydrogen peroxide (H2O2). H2O2 can decompose exothermically to water and oxygen gas by the following stoichiometry: 2H2O2 [symbol] 2 H2O+O2. This reaction can occur spontaneously but slowly over time. The addition of an activator catalyst (e.g., catalase, peroxidases, manganese dioxide, iron, and many others) can substantially accelerate the reaction.

In one example, the O2 donor can be a superoxide, e.g., potassium superoxide (KO2). KO2 can react exothermically with water to produce KOH and oxygen, as illustrated in the following equilibrium reaction: 4 KO2+2 H2O [symbol] 4 KOH+3 O2. In one example, any compound which is capable of releasing O2 upon reaction with an activator can be suitable as a O2 donor.

### Carbon Monoxide

In one example, carbon monoxide, having the molecular formula CO, can be a very small molecule that, upon release from a composition, can migrate fast and reach its target site. It can exhibit biological properties which are similar to NO, as described, for example, in Korean J Intern Med. 2013; 28:123-40. Thus, in one example, the composition of the present invention can contain a CO-donor and an activator that induces the release of CO.

### Additional Therapeutic Agent

In one example, several conditions can involve a combination of etiological factors, some of which can be affected by the biologically-active gas; other etiological factors can have an additional therapeutic modality. For example, psoriasis may be treated by nitric oxide (a biologically-active gas), as well as a steroid drug, and therefore combined treatment would be beneficial. Likewise, acne, which can involve a microbial infection, excessive keratin production, excessive sebum production and inflammation, can benefit from treatment with a combination of oxygen or nitric oxide (biologically-active gases), and an additional therapeutic agent, selected from the group consisting of an anti-inflammatory agent, an antibiotic agent, a sebostatic agent and a keratolytic agent. Hence, in many cases, the inclusion of an additional therapeutic agent in the composition can contribute to the clinical activity of the biologically-active gas.

Suitable additional therapeutic agents can include but are not limited to active herbal extracts, acaricides, age spot and keratose removing agents, allergen, analgesics, antiacne agents, antiallergic agents, antiaging agents, anti-bacterials, antibiotics, antiburn agents, anticancer agents, antidandruff agents, antidepressants, anti-dermatitis agents, anti-edemics, antihistamines, anti-helminths, anti-hyperkeratolyte agents, anti-inflammatory agents, anti-irritants, anti-lipemics, antimicrobials, antimycotics, antiproliferative agents, antioxidants, anti-wrinkle agents, anti-pruritics, anti-psoriatic agents, anti-rosacea agents anti-seborrheic agents, antiseptic, anti-swelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, local anesthetics, metal oxides, mitocides, neuropeptides, non-steroidal anti-inflammatory agents, oxidizing agents, pediculicides, photodynamic therapy agents, retinoids, scabicides, self-tanning agents, skin whitening agents, vasoconstrictors, vasodilators, vitamins, vitamin D derivatives, wound healing agents, and wart removers. Some instances a specific active agent may have more than one activity, function or effect.

### Propellants

In one example, aerosol propellants can be used to generate a positive pressure inside the gas-resistant container. The positive pressure can be useful to inhibit the production of the biologically-active gas; and to facilitate dispensing the composition from the container.

In one example, a positive pressure can be imposed by adding a propellant. In certain cases, the propellant can be added directly into the container that contains the gas-evolving composition. In the case of a “bag on valve”, a “bag-in-can”, a “piston can” or a “can-in-can” aerosol container, the propellant can be added into the space 108 between the external container and the internal container, as illustrated in FIG. 1d; and in certain cases, the propellant can be added into both the space 108 between the external container and the internal container and into the inner container 106 that contains the gas-evolving composition.

In one example, the propellant can be a liquefied gas (i.e., a liquid that is above its boiling point at normal room temperature). Upon adding a propellant into the container, the space above the liquid can fill with vapor because the liquid can be trying to boil. As the product is used, the liquid propellant can evaporate to fill the space and keep the pressure constant. Examples of suitable liquefied gas propellants can include volatile hydrocarbons such as butane, propane, isobutane and fluorocarbon gases, or mixtures thereof. In one example, a compressed gas, such as air, nitrogen, helium, argon and CO2 can be used as a propellant.

### Forms of the Composition

In one example, the gas-releasing composition can comprise a vehicle, a gas donor and an activator. While the composition may have various rheological characteristics, the following non-limiting examples of forms of the composition are provided for demonstration purposes.

In one example, the composition (e.g. NORS) can be a liquid. The liquid is administered to the application situs (e.g. site of a wound or manifestation of symptoms of a disease or condition) and nitric oxide is released from the composition at the site. A liquid composition can be flowable. The liquid can be dispensed from the packaging system upon opening the valve, in the presentation of a flowable liquid. If the actuator is suitable of spraying a liquid, the composition can be released in the form of a spray. If the valve or the actuator is suitable for releasing a controllable amount of a composition, a controlled amount of liquid or spray can be dispensed. Depending on the selection of nozzle for the container, the liquid composition can be dispensed in any specific pattern or amount. For example, wide spray patterns, medium spray patterns, focused spray patterns (e.g. streams) of liquid or liquid droplets can be achieved, as well as fogs, mists or other particulate arrangements.

In one example, the composition can be an aqueous liquid, wherein the gas donor and the activator are either in solution or in suspension. In one example, the composition can be a semi-solid. In one example, the composition can have a viscosity of more than about 5,000 Cps and it can have a viscosity selected from the group of: between about 5,000 Cps and about 100,000 Cps; between about 5,000 Cps and about 20,000 Cps; between about 20,000 Cps and about 60,000 Cps; and between about 60,000 Cps and about 100,000 Cps.

In one example, the composition can be a gel. The viscosity of the gel can be attained using customary polymeric or gelling agents. Exemplary polymeric or gelling agents include, in a non-limiting manner, naturally-occurring polymeric materials, such as locust bean gum, sodium alginate, sodium caseinate, egg albumin, gelatin agar, carrageenan gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinyl pyrrolidone, polyvinyl alcohol, polyacrylic acid polymers, polymethacrylic acid polymers, polyvinyl acetate polymers, polyvinyl chloride polymers, polyvinylidene chloride polymers, and the like.

Additional exemplary polymeric agents can include semi-synthetic polymeric materials such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, and cationic celluloses. Polyethylene glycol, having molecular weight of 1000 or more (e.g., PEG 1,000, PEG 4,000, PEG 6,000 and PEG 10,000) can also have gelling capacity and while considered herein as “secondary polar solvents”, as detailed herein, PEG can also be polymeric agents. The foregoing polymeric agents can be mixed.

In one example, the concentration of the polymeric agent can be selected so that the composition has the desirable viscosity. In one example, the concentration of the polymeric agent can be selected such that the viscosity of the composition, prior to filling of the composition into aerosol canisters, can be more than about 5,000 Cps and it can have a viscosity selected from the group of: between about 5,000 Cps and about 100,000 Cps; between about 5,000 Cps and about 20,000 Cps; between about 20,000 Cps and about 60,000 Cps; or between about 60,000 Cps and about 100,000 Cps.

In one example, the composition can be an aqueous gel (i.e., a gel that contains water) wherein the gas donor and the activator can be either in solution or in suspension. In one example, the aqueous gel can comprise water and a polar solvent. In one example, the composition can be an emulsion, or micro-emulsion, or a nano-emulsion, which can include an aqueous phase and an organic carrier phase. Examples of pharmaceutical dosage forms that comprise an emulsion can include creams, lotions and emulsion-based sprays and foams.

In one example, the organic carrier can be selected from a hydrophobic organic carrier (also termed herein “hydrophobic carrier”), an emollient, a polar solvent, and mixtures thereof. A “hydrophobic organic carrier” as used herein can refer to a material having solubility in distilled water at ambient temperature of less than about 1 gm per 100 mL, less than about 0.5 gm per 100 mL, or less than about 0.1 gm per 100 mL.

In one example, the hydrophobic carrier can be an oil, such as mineral oil. According to one example, hydrophobic carriers can be oils originating from plant, marine or animal sources. By way of example, the plant oil may be olive oil, corn oil, soybean oil, canola oil, cottonseed oil, coconut oil, sesame oil, sunflower oil, borage seed oil, Syzigium aromaticum oil, hempseed oil, herring oil, cod-liver oil, salmon oil, flaxseed oil, wheat germ oil, evening primrose oils or mixtures thereof, in any proportion.

In one example, suitable hydrophobic carriers can also include polyunsaturated oils that contain for example omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Such unsaturated fatty acids can have a skin-conditioning effect, which can contribute to the therapeutic benefit of the biologically-active gas. In one example, oils that possess therapeutically-beneficial properties are termed “therapeutically active oil”.

In one example, silicone oils can also be used. Suitable silicone oils can include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These can be chosen from cyclic or linear polydimethylsiloxanes containing from about 3 to about 9, or about 4 to about 5, silicon atoms. Volatile silicones such as cyclomethicones can also be used. Silicone oils are also considered therapeutically active oil, due to their barrier retaining and protective properties.

In one example, the organic carrier can contain a mixture of two or more of the above hydrophobic carriers in any proportion. A further class of organic carrier can include “emollients” that can have a softening or soothing effect, especially when applied to body areas, such as the skin and mucosal surfaces. Emollients may not be hydrophobic. Examples of suitable emollients can include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate, and mixtures thereof. In one example, a “polar solvent” can be an organic solvent, typically soluble in both water and oil. Examples of polar solvents can include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, such as dimethylsulfoxide (DMSO), dimethylformanide, methyl dodecyl sulfoxide, dimethylacetamide, monooleate of ethoxylated glycerides (with 8 to 10 ethylene oxide units), azone (1-dodecylazacycloheptan-2-one), 2-(n-nonyl)-1,3-dioxolane, and admixtures thereof.

In one example, surface-active agents (also termed “surfactants”) can include any agent linking oil and water in the composition in the form of emulsion. A surfactant's hydrophilic/lipophilic balance (HLB) describes the emulsifier's affinity toward water or oil. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average).

In one example, the surface-active agent can have a hydrophilic lipophilic balance (HLB) between about 9 and about 14, which is the HLB to stabilize an O/W emulsion of a given oil of hydrophobic carriers or oils. Thus, in one example, the composition can contain a single surface-active agent having an HLB value between about 9 and 14, and in one example, the composition can contain more than one surface active agent and the weighted average of their HLB values can be between about 9 and about 14. In one example, when a water in oil emulsion is desirable, the composition can contain one or more surface active agents, having an HLB value between about 2 and about 9.

In one example, the surface-active agent can be selected from anionic, cationic, nonionic, zwitterionic, amphoteric and ampholytic surfactants, as well as mixtures of these surfactants. Nonlimiting examples of possible surfactants can include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49, Myrj 52 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; mono or diglycerides, isoceteth-20, sodium methyl cocoyl taurate, sodium methyl oleoyl taurate, sodium lauryl sulfate, triethanolamine lauryl sulfate, and betaines.

In one example, the surface-active agent can include at least one non-ionic surfactant. Ionic surfactants can be irritants. Therefore, non-ionic surfactants can be used in applications including sensitive tissue such as found in most mucosal tissues, especially when they are infected or inflamed. In one or more embodiments, the surface-active agent can include a mixture of at least one non-ionic surfactant and at least one ionic surfactant in a ratio in the range of about 100:1 to 2:100. In an additional embodiment the gas evolving composition can provide a foam upon dispensing from the pressure-resistant container.

### Treatment/Therapy

In one example, the terms “therapy” and “treatment” can be used interchangeably, to cover any treatment of a disease or disorder, and can includes, for example: curing the disease or disorder; preventing the disease or disorder from occurring in a subject which may be predisposed to the disease but has not yet been diagnosed as having it; inhibiting the disease or disorder; relieving the disease or disorder; causing regression of the disease; providing a beneficial immunological effect; improving the quality of life of a subject afflicted by a disease or disorder; and, in the case of cosmetic treatment cleansing, beautifying, promoting attractiveness, or altering the appearance without affecting the body's structure or functions.

### Fields of Pharmaceutical Applications

In one example, by including an appropriate gas donor, which can evolve a biologically-active gas, the composition can treat a patient having any one of a variety of dermatological disorders (also termed “dermatoses”), such as classified, in a non-limiting exemplary manner, according to the following groups: dermatitis, including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash; bacterial infections, including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma; fungal infections, including dermatophyte infections, yeast Infections; parasitic infections, including scabies, pediculosis, creeping eruption; viral infections; disorders of hair follicles and sebaceous glands, including acne, rosacea, perioral dermatitis, hypertrichosis (hirsutism), alopecia, male pattern baldness, alopecia areata, alopecia universalis and alopecia totalis; pseudofolliculitis barbae, keratinous cyst; scaling papular diseases, including psoriasis, Pityriasis rosea, lichen planus, Pityriasis rubra pilaris; benign tumors, including moles, dysplastic nevi, skin tags, lipomas, angiomas, pyogenic granuloma, seborrheic keratoses, dermatofibroma, keratoacanthoma, keloid; malignant tumors including basal cell carcinoma, squamous cell carcinoma, melanoma, paget's disease of the nipples, kaposi's sarcoma; reactions to sunlight, including sunburn, chronic effects of sunlight, photosensitivity; bullous diseases, including pemphigus, bullous pemphigoid, dermatitis herpetiformis, linear immunoglobulin A disease; pigmentation disorders, including hypopigmentation, vitiligo, albinism, post-inflammatory hypopigmentation, post-inflammatory hyperpigmentation, melasma, chloasma, drug-induced hyperpigmentation; disorders of cornification, including ichthyosis, keratosis pilaris, calluses, corns, actinic keratosis; pressure sores; disorders of sweating; inflammatory reactions including drug eruptions, toxic epidermal necrolysis; erythema multiforme, erythema nodosum, or granuloma annulare.

In one example, the compositions can treat non-dermatological disorders, which can respond to topical or transdermal delivery of an active agent. By way of example, such disorders can include localized pain and/or in general, as well as joint pain, muscle pain, back pain, rheumatic pain, arthritis, osteoarthritis and acute soft tissue injuries and sports injuries. Other disorders of this class can include conditions, which respond to hormone therapy, such as hormone replacement therapy, transdermal nicotine administration, and other respective disorders. These examples, the biologically-active gas can increase the peripheral blood pressure or increase dermal blood flow for diseases or disorders associated with decreased peripheral blood pressure or blood flow, such as Raynaud's syndrome.

In one example, the compositions of the present invention can be used for the treatment and prevention of disorders and diseases of other body areas and cavities including the skin, mucosal membranes, the anum, the rectum, the GI system, the vagina, the penile urethra, the eye, the respiratory system, including the oral cavity, the nasal cavity, the sinuses, the pharnix, the larynx, the trachea, the bronchus and the lungs, the dental system, and the ear canal.

In one example, the compositions can treat a patient having any one of a variety of gynecological disorders, such as classified, in a non-limiting exemplary manner, according to the following groups: pelvic pain, including premenstrual syndrome (PMS), mittelschmerz (severe midcycle pain due to ovulation), dysmenorrhea (pain related to the menstrual cycle), endometriosis, ectopic pregnancy, ovarian cysts and masses, acute pelvic inflammatory disease, pelvic congestion syndrome and vulvodynia; vulvovaginal infections, including bacterial vaginosis, candidal vaginitis, Trichomonas vaginalis, herpes simplex genital ulcers and warts, pelvic inflammatory disease, cervicitis, acute and chronic salpingitis; endometriosis; gynecological neoplasms, including endometrial Cancer, ovarian cancer, cervical cancer, vulvar cancer, vaginal cancer, fallopian tube cancer and gestational trophoblastic disease; benign tumors; sexually transmitted diseases; sexual dysfunction disorders that respond to pharmacological therapy, including sexual arousal disorder, female orgasmic disorder, dyspareunia and vaginismus; and various gynecological disorders that respond to hormonal therapy.

In one example, rectal applications can include, for example, anal abscess/fistula, anal cancer, anal warts, hemorrhoids, anal and perianal pruritus, soreness, excoriation, perianal thrush, anal fissures, fecal incontinence, constipation, Crohn's disease, inflammatory Bowel's disease and polyps of the colon and rectum.

In one example, the compositions can be useful for intra-vaginal and rectal treatment of sexually-transmitted and non-sexually-transmitted infectious disease (STDs). In one example, a method of treatment can include treating a disease or disorder of the skin, mucosal membranes, the anum, the rectum, the GI system, the vagina, the penile urethra, the eye, the respiratory system, including the oral cavity, the nasal cavity, the sinuses, the pharnix, the larynx, the trachea, the bronchus and the lungs, the dental system, and the ear canal, comprising topical administration of the composition, whereby one or more biologically-active gases, in a therapeutically effective concentration can be administered topically to the afflicted area.

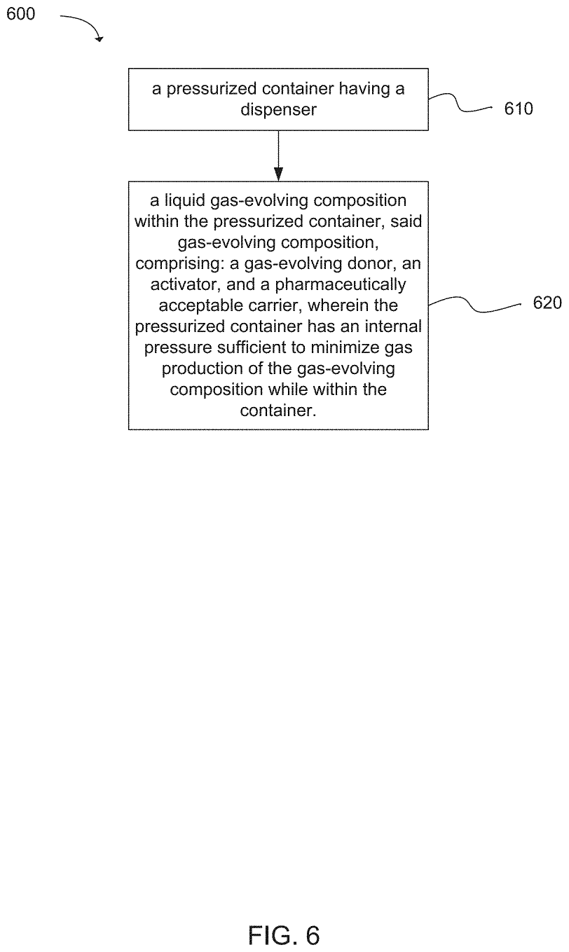

In one example, as depicted in the flowchart in FIG. 6, a gas-evolving composition delivery system can comprise a pressurized container having a dispenser, as sin block 610. The pressurized container can further comprise a liquid gas-evolving composition within the pressurized container, said gas-evolving composition, comprising: a gas-evolving donor, an activator, and a pharmaceutically acceptable carrier, wherein the pressurized container has an internal pressure sufficient to minimize gas production of the gas-evolving composition while within the container, as in block 620.

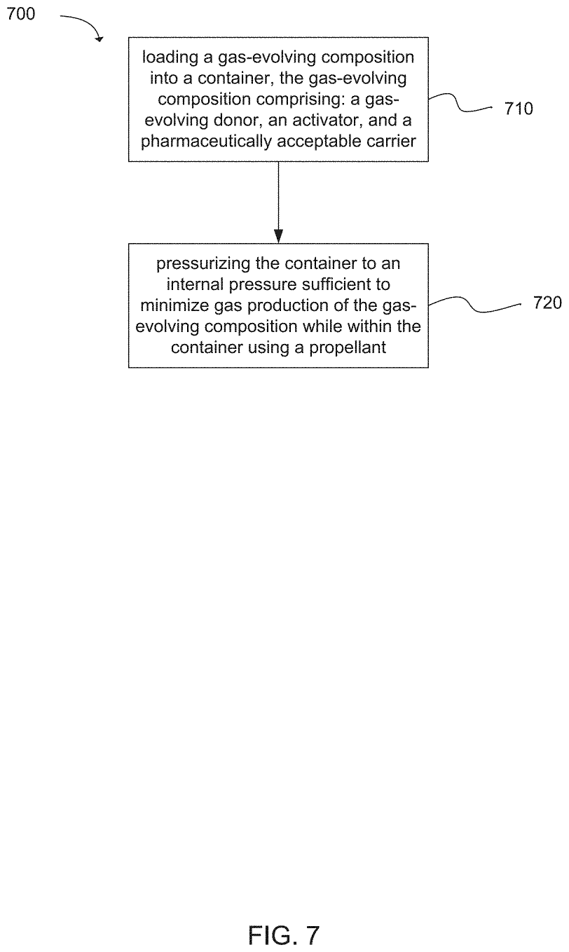

In one example, as depicted in the flowchart in FIG. 7, a method of stabilizing an acidified nitrite solution can comprise: loading the acidified nitrite solution into a container, the acidified nitrite solution comprising: a nitric oxide (NO) evolving donor; an NO activator; and a pharmaceutically acceptable carrier, as in block 710. The method can further comprise sealing the container to be airtight, as in block 720.

In one example the container can be sealed with nitrite solution and then the acid concentrated solution can be inserted. In another example the container can be sealed with acid concentrated solution in it and then the nitrite solution can be inserted.

In one example, the method can further comprise: reducing a headspace in the container to below a predetermined threshold amount. The predetermined threshold amount can include the threshold amount of headspace is less than: 5% of the container, 4% of the container, 3% of the container, 2% of the container, 1% of the container, 0.5% of the container, 0.1% of the container, or 0.01% of the container. The method can further comprise: reducing an amount of oxygen in a headspace of the container below a predetermined threshold. The threshold amount of oxygen in the head space of the container can be less than: 21%, 5%, 2%, 1%, or 0.1%. In additional examples, the headspace may be filled with an element that is inert or non-reactive with the other components of the container, such as nitrogen gas.

In one example, the acidified nitrite solution can be configured to maintain minimum threshold donor and activator concentrations over a predetermined period of time. The threshold concentrations can be at least one of: a minimum threshold donor concentration of 2 millimolar (mM), 10 mM, 50 mM, 100 mM, 200 mM, or 500 mM; or a minimum threshold activator concentration with a pH of 2.8-5.5. The period of time can be at least one of: 7 days, 14 days, 30 days, or 60 days.

In one example, the NO-evolving donor can comprise a nitrite or nitrite salt, a nitrate or nitrate salt, sodium nitroprusside, molsidomine or a metabolite thereof, a diazeniumdiolates, a S-nitrosothiol, mesoionic oxatriazole, iron-sulphur nitrosyls, sinitrodil, a NONOate compound, or a combination thereof.

In one example, the NO activator can comprise a metal, a hydroxy acid, citric acid, ascorbic acid, lactic acid, glycolic acid, salicylic acid, hydrochloric acid, nitric acid, sulfuric acid, phosphoric acid, sodium iodide, iron sulfate, potassium nitrate, a chromate salt, or a combination thereof. In one example, the pharmaceutically acceptable carrier can be at least one of an aqueous liquid or a hydrophobic carrier. In one example, the acidified nitrite solution can further comprise a gelling agent, a polymeric agent, an emollient, a polar solvent, a surface-active agent, an oil, or a combination thereof. In one example, the acidified nitrite solution can have a pH of from about 1 to about 7.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1—Stability of Nitric Oxide Evolving Composition

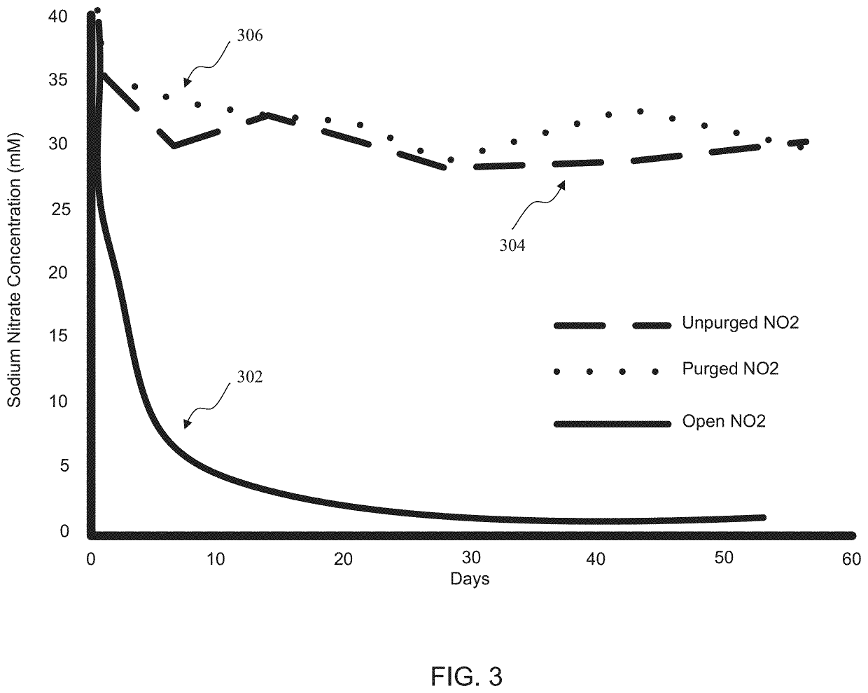

This experiment demonstrates that upon storage in a pressure-resistant container, a nitric oxide (NO)—releasing composition remained stable for an extended period of time. The NO-releasing composition comprised a solution of sodium nitrite 40 mM (NO-donor); and a citric acid (activator) added to effectuate a pH=3.5. The NO-releasing composition was placed into a container with a crimp-on aluminum cap up to the top in which the volume of the container was about 55 mL. The cap was then crimped onto the container. The control system comprised the same container filled with 40 mM nitric oxide solution without crimping the cap. The experiment was conducted twice, with and without purging the NO-releasing solution with nitrogen. Samples were taken from the containers throughout a period of 56 days (8 weeks). FIG. 3 illustrates that the crimped solutions, with purging 306 or without purging 304, were equally stable, while the non-crimped (open) solutions 302 degraded rapidly.

### Example 2—Preparation of a Nitric Oxide Evolving Composition in an Aerosol can

The following experiment demonstrates the preparation of a nitric oxide (NO)—releasing compositions in aerosol cans. 1000 ml of Nitric Oxide donor solution was prepared by dissolving the following amounts of sodium nitrite (NaNO2) in purified water, as depicted in Table 1:

TABLE 1

| | |
|---|---|
| Target Sodium Nitrate | Sodium Nitrite |
| Concentration | (gr.) |

| | | | |
|---|---|---|---|
| Solution A | 20 | mM | 1.38 |
| Solution B | 60 | mM | 4.14 |
| Solution C | 100 | mM | 6.90 |

44 gr of each solution was added to 38×107 mm aluminum canister, with PAM lining. The fill capacity of a can was approximately 100 ml. The cans were cooled in an ice bath. The following amounts of activator citric acid were added to each can based on the target sodium nitrate concentration, as depicted in Table 2:

TABLE 2

| | |
|---|---|
| Target Sodium Nitrate | Sodium Nitrite |
| Concentration | (gr.) |

| | | | |
|---|---|---|---|
| Solution A | 20 | mM | 0.6 |
| Solution B | 60 | mM | 0.18 |
| Solution C | 100 | mM | 0.30 |

Each can was flushed with nitrogen, and crimped immediately with an aerosol valve (Aptar). Hydrocarbon propellant AP70 (approximately 35 cc) was then added through the valve to achieve a ratio of 70% nitric oxide evolving composition/30% propellant.

### Example 3—Determination of the Stability of NORS at Varying Concentrations and Volumes

Nitric oxide releasing solution (NORS) was used in various treatment experiments. The preparation model in use was to mix and activate on site requiring an available pH meter or a previously determined mass of citric acid for activation. However, when varying concentrations and volumes, depending on the treatment in development, this could be cumbersome and time consuming. Preparing a solution ahead of time was used when needed.

### Procedure

### Preparing NaNO2 Standards

Using a Mettler AC100 balance, a KIMAX 200 mL volumetric flask, isotonic saline, and Sodium Nitrite from Sigma-Aldrich, standards were prepared to desired concentrations: 20 mM, 40 mM, and 100 mM. Standards were stored temporarily in a 500 mL Erlenmeyer flask until being transferred into bottles used for long term storage. There were two types of bottles 50 mL serum bottles with crimp-on aluminum caps from Supelco and 20 mL disposable scintillation vials from VWR with caps modified to be used with rubber stoppers. The bottles were filled to the bottom of the neck resulting in 55 mL total volume for the serum bottles and 21 mL for the vials.

### Determining Activation Concentrations:

### 20 mM NORS:

For determining the moles of citric acid used to activate the 20 mM standards, tubes with 40 mL of 20 mM NaNO2 were activated with 1 M citric acid until the solution reached pH 3.5. The pH was measured with an Orion Star A211 pH meter from Thermo Scientific and the process was repeated until the pH of the 20 mM solution could be lowered with one addition of acid. This process was repeated with 60 mM Sodium Nitrite and results were compared to determine a molar ratio used to lower the pH of the standards to 3.5. Using the determined molar ratio five 55 mL and five 21 mL samples of 20 mM sodium nitrite were prepared for activation in the 50 mL serum bottles and 20 mL vials respectively. Starting 50 uL below the desired activation volume and increasing in 25 uL increments, volumes of 1 M citric acid were added to each sample to further refine the volume to create the pH 3.5 NORS samples. This extra step was taken to ensure as little interference with the samples once the experiment had started, and so the samples could be activated with one confident injection.

### 100 mM NORS:

Using the molar ratio determined from the 20 mM and 60 mM tests, a similar process to the above method was used to determine the activation volume to lower the pH of a 100 mM sodium nitrite solution to pH 3.5. For this test, the bottles and vials were filled with 55 mL and 21 mL of 100 mM nitrite standard respectively to account for the addition of 1 mL of citric acid and the acid was prepared to meet the molar ratio. Slightly varying volumes were used to activate a set of bottles before the pH was tested and the optimal injection was chosen to activate the bottles for the experiment. Concentrations and volumes chosen were 310 uL of 1.61 M citric acid for the 21 mL samples and 740 uL of 1.77 M citric acid for the 55 mL samples to drop their pH to 3.5.

### 40 mM NORS:

With combined data from the 20 mM and 100 mM activation tests, a molar ratio was determined to activate the 40 mM sodium nitrite standards to pH 3.5. A 40.741 mM solution was prepared to account for the change in concentration after activation and citric acid was prepared to the desired concentration to activate the solution with a 1 mL injection. The tests were only done in 50 mL bottles since the 20 mL vials were omitted from this part of the experiment. The 54 mL samples of 40.741 mM nitrite were activated and the concentration of the citric acid was adjusted until the samples could be activated to a final concentration of 40 mM nitrite in 55 mL at pH 3.5. The concentration of citric acid to use 1 mL to activate 54 mL of 40.741 mM sodium nitrite was 0.555 M.

### Purging of Standards:

### 20 mM Purged NaNO2:

Four 50 mL bottles and four 20 mL bottles were filled with 55 mL and 21 mL of 20 mM NaNO2 respectively. The four 50 mL bottles and four 20 mL bottles were sealed with a chlorobutyl rubber stopper and aluminum crimp seal and then brought to a compressed N2 tank to be purged of their oxygen content. This was done to prevent the oxidation of NO2- to NO3- over time in the solution. Purging was completed by inserting two needles into the bottles. An 18 gauge 1½ inch needle connected to the compressed N2 tank was inserted through the stopper and submerged in the liquid. The second 26 gauge and ⅜ inch needle was inserted to prevent pressure build up. Using an air flow meter the N2 flow was set to 4 L/min and the 50 mL bottles were purged for 15 min followed by the 20 mL vials being purged for 10 min. After purging the bottles were stored inside a lidded cardboard box to block out light.

### 100 mM and 40 mM NaNO2 Purging:

Four 50 mL and four 20 mL bottles were filled with 55 mL and 21 mL of 100 mM NaNO2 respectively. The 50 mL bottles were sealed with chlorobutyl rubber stoppers and an aluminum crimp seal, and the 20 mL vials were sealed with a rubber stopper and screw cap modified with a hole for syringe insertion. The purge procedure was the same as the 20 mM NaNO2 except the purge time for the 50 mL bottles was reduced to 5 min and the 20 mL purge time was reduced to 2 min. The 40 mM NaNO2 samples were purged similar to the 100 mM samples except that the 20 mL vials were omitted.

### Activation of Samples and Measurement Scheme

### 20 mM Purged:

Samples were prepared in two sets of eight bottles, four 50 mL and four 20 mL. The first set was activated with 900 uL of 0.375 M citric acid and 400 uL of 0.3 M citric acid respectively for 55 mL and 21 mL samples with a 1 mL disposable syringe. The syringe was rinsed first with the acid and bubbles were pumped out before the activation injection was delivered. Measurements for the first set were quenched by dilution and measured as follows. A 15 mL tube, one for each sample bottle, was filled with 9.9 mL of DIH2O using a power pipette and 10 mL serological pipette and then 100 uL of sample was added to the tube using a 100 uL analytical syringe. The tube was mixed via vortex or shaking and brought to the Chemi for analysis with the Vanadium (III) Chloride reducing agent. This process was repeated at 30 min, 1 hour, 2 hours, 4 hours, and 8 hours on the first day. Follow up measurements were taken after 24 hours, 48 hours, 4 days, 7 days, 14 days, 21 days, 12 weeks, 18 weeks and 22 weeks. At the 12-week measurement the dilutions were changed to 8 mL of DIH2O with 100 uL of sample in a 15 mL tube after the acquisition of a volumetric pipette.

The second set of 20 mM samples was prepared in the same way with only the activation injection for the 50 mL bottles increasing to 950 uL of 0.375 M citric acid. The second set was analyzed with the sodium iodide reducing agent and the Chemi following the same measurement plan, but starting the day after the first set was activated.

The pH measurements started on the 10th day after activation due to the lack of a pH meter that could measure from a 200 uL sample. Samples for pH measurement were extracted with a disposable 26 gauge ⅜ inch needle and 1 mL syringe, and they were measured with a HORIBA LAQUA 713 hand held pH meter using a 2 point calibration before each set of measurements. The extractions for measurement were approximately 200 uL in size and this measurement scheme was similar over all concentrations used in the experiment.

### 100 mM Unpurged and Purged:

Samples were prepared similar to the 20 mM sets. Unpurged samples were prepared by adding 55 mL and 21 mL of 100 mM NaNO2 into 50 mL and 20 mL bottles respectively. The unpurged bottles were closed with a rubber stopper and an aluminum crimp seal, and activated immediately. They were activated with 740 uL of 1.77 M citric acid for the 50 mL bottles and 310 uL of 1.61 M citric acid for the 20 mL vials. Purged samples were prepared and purged as described in the above section on purging. They were activated with 770 uL of 1.70 M and 321 uL of 1.55 M citric acid for the larger and smaller bottles respectively. In this case the activation injections varied from the unpurged set in order to match the concentration of the two sets since the pH of the solution seems more dependent on the NaNO2 concentration than the volume. Final concentration of the 55 mL samples was 99.19 mM, and 99.06 mM was the 21 mL samples final concentration.

Day 0 nitrite and nitrate extractions for measurement were quenched within the first hour after activation by dilution by combining 8 mL of DIH2O and 50 uL of sample to reach a concentration within the range of the standard curve used by the chemi and were measured as is outlined in the SOPs for the chemi. Measurement procedures were repeated on days 1, 2, 4, 7, 14, 21, 28, 42, 56, and 84. The sample's pH was measured on the same day as the nitrites and nitrates were measured.

### 40 mM Unpurged, Purged and Open System:

As was earlier stated the 40 mM samples were only prepared in 50 mL bottles. They were prepared in sets of 5 bottles and activated on three separate days. Unpurged bottles were prepared by adding 54 mL of 40.741 mM NaNO2 to the 50 mL bottles and closing them with a rubber stopper and aluminum crimp seal before activating. The purged set was prepared the same as the unpurged bottles except purged as described above. The open system set was prepared the same way as the other two sets, but the stopper and crimp seal were omitted. All sets were activated with a 1 mL injection of 0.555 M Citric acid. The resulting final solution was a 40 mM NaNO2 solution at pH 3.5.

These sets were all measured within one hour of their activation on Day 0, and measurements were repeated on days 1, 3, 7, 14, 21, 28, 42, and 56. For measurement the samples were quenched by diluting 10 uL of sample solution, extracted via a 100 uL VICI fixed needle analytical syringe, in 790 uL of DIH2O. The pH was also measured on the same day of extraction.

### Results and Observations

### 20 mM Purged

The first observation from the measurement of the samples is that dilutions take about 15 min. This meant that the 30 min extractions could not be measured until after the 1-hour extractions had also been diluted. Measuring eight samples with the Chemi took about 30 min, and so the measurements were not taken at the exact time interval indicated. After the first week the rubber stoppers appeared swollen and lighter in color and small parts of the stopper had fallen into the solution likely from insertion of the needle. Samples remained clear and colorless over the duration of the experiment.

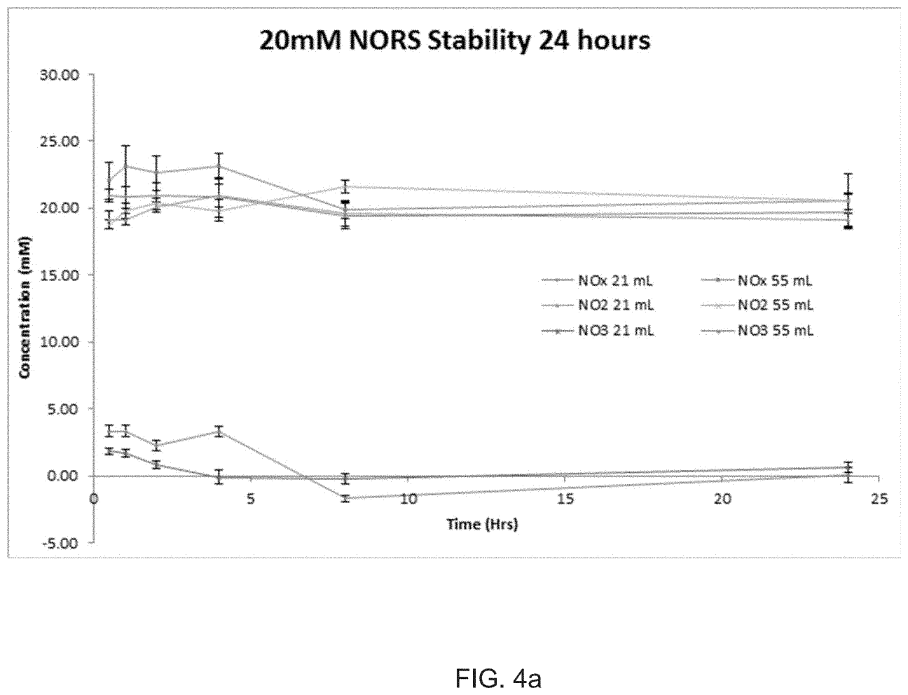

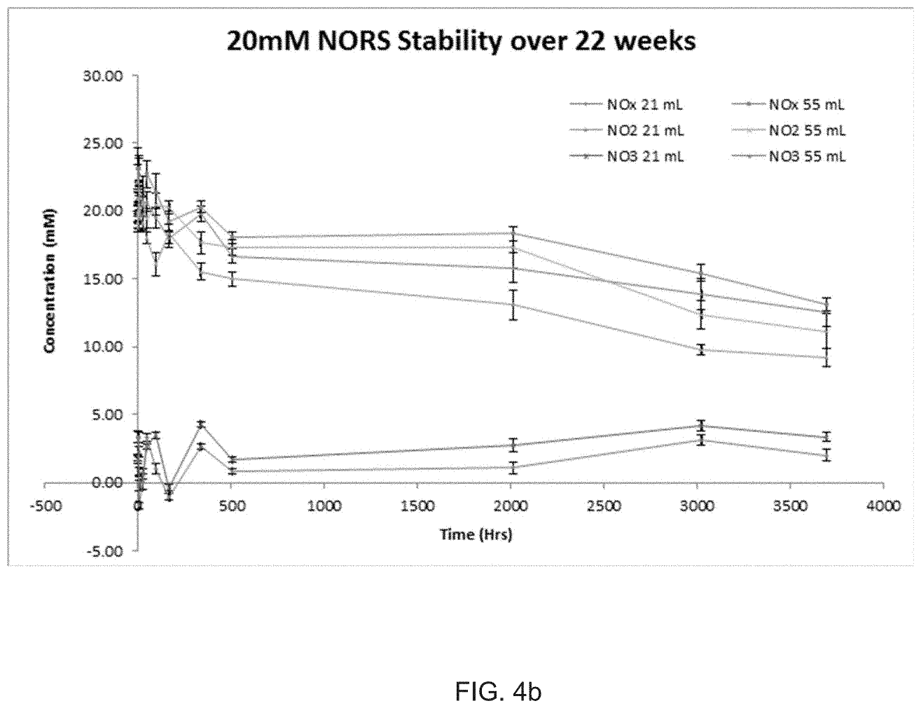

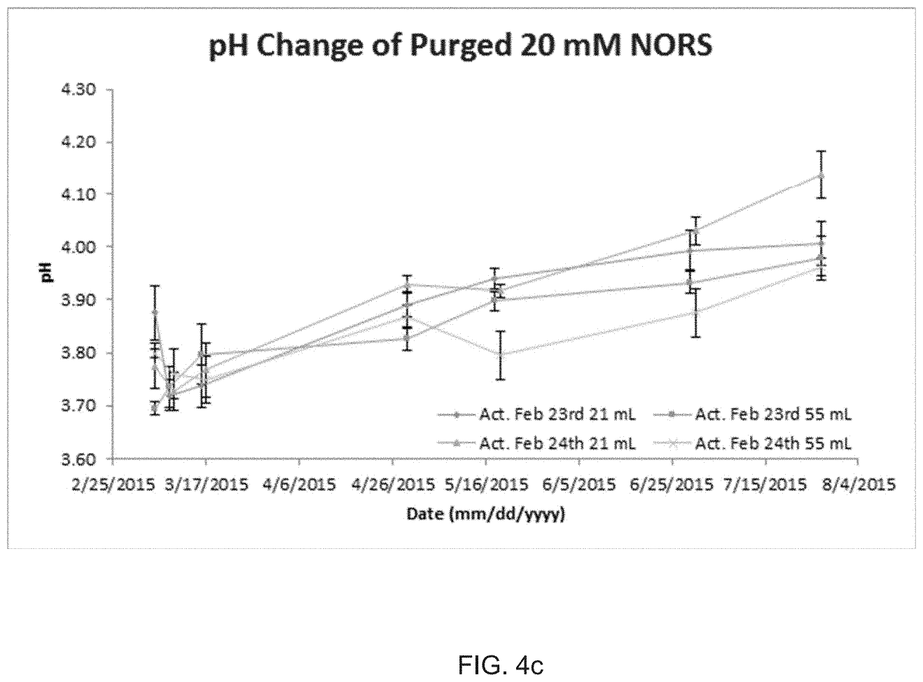

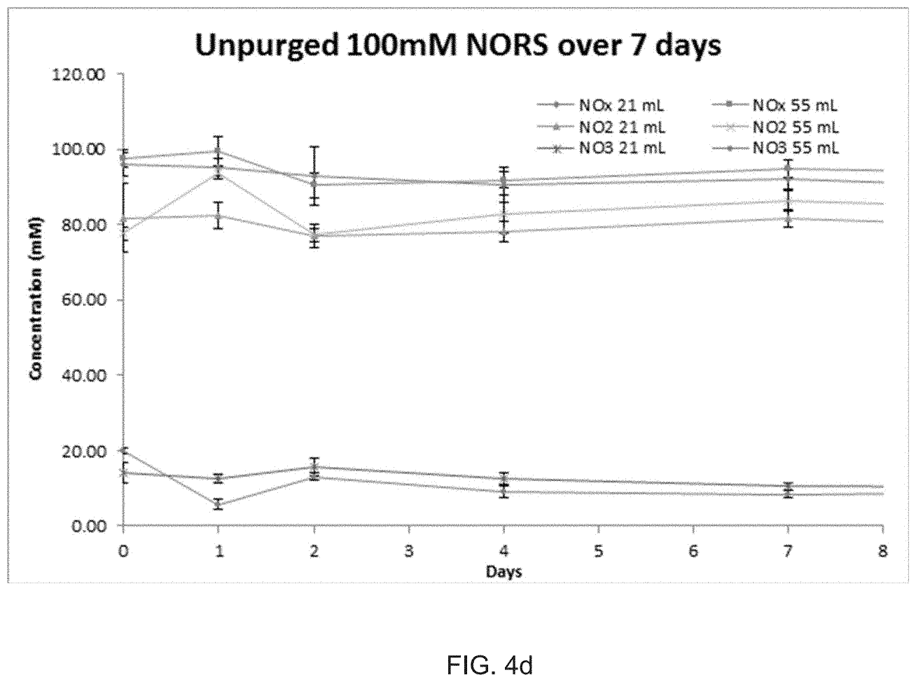

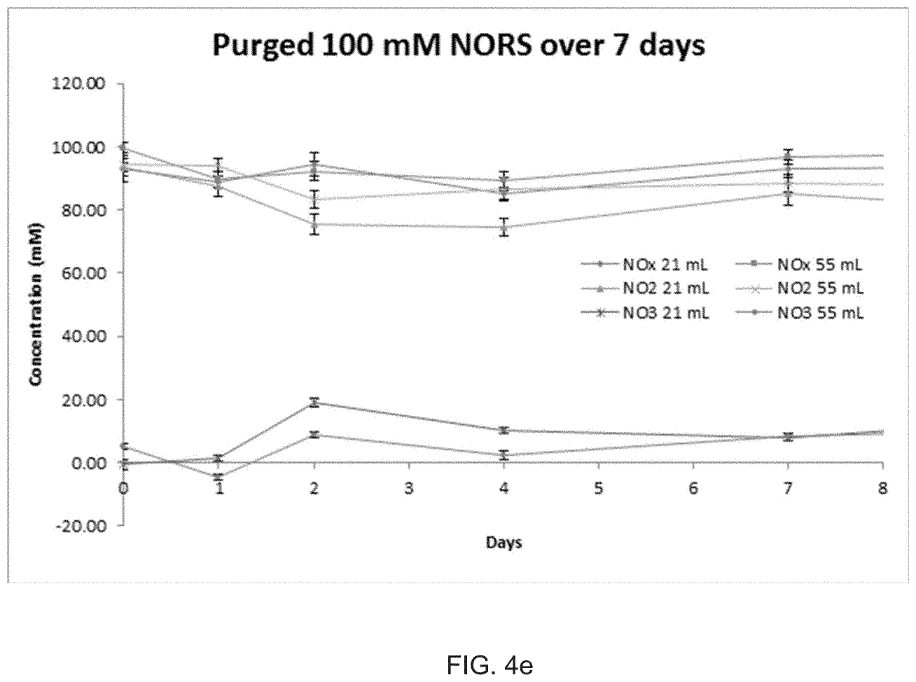

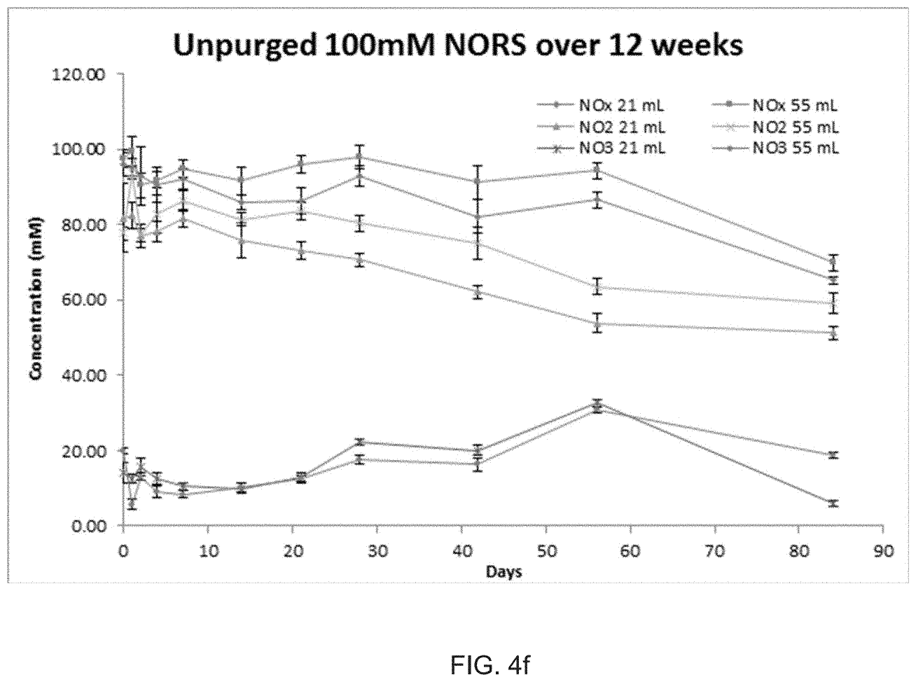

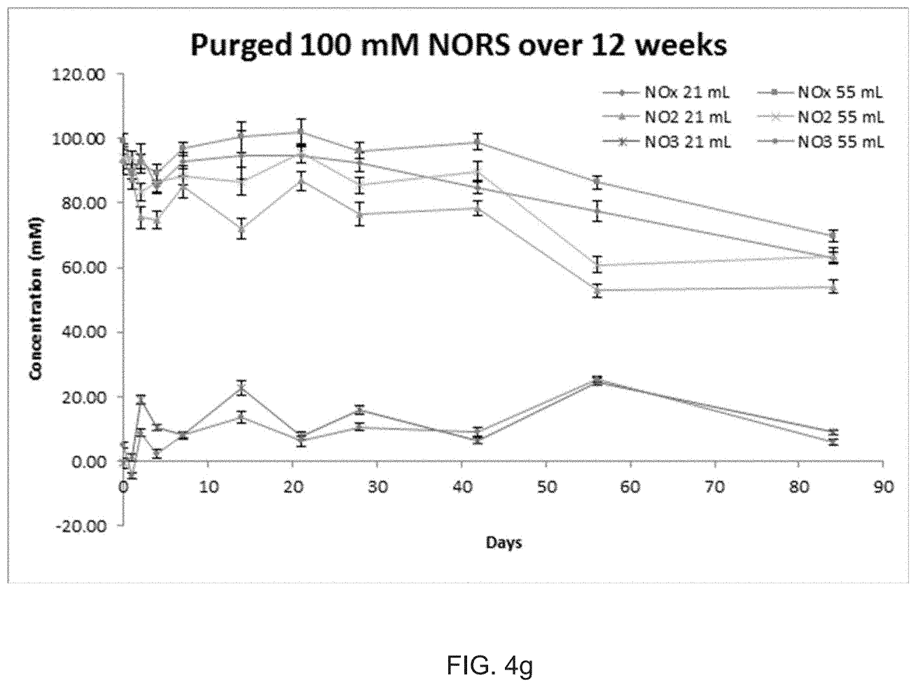

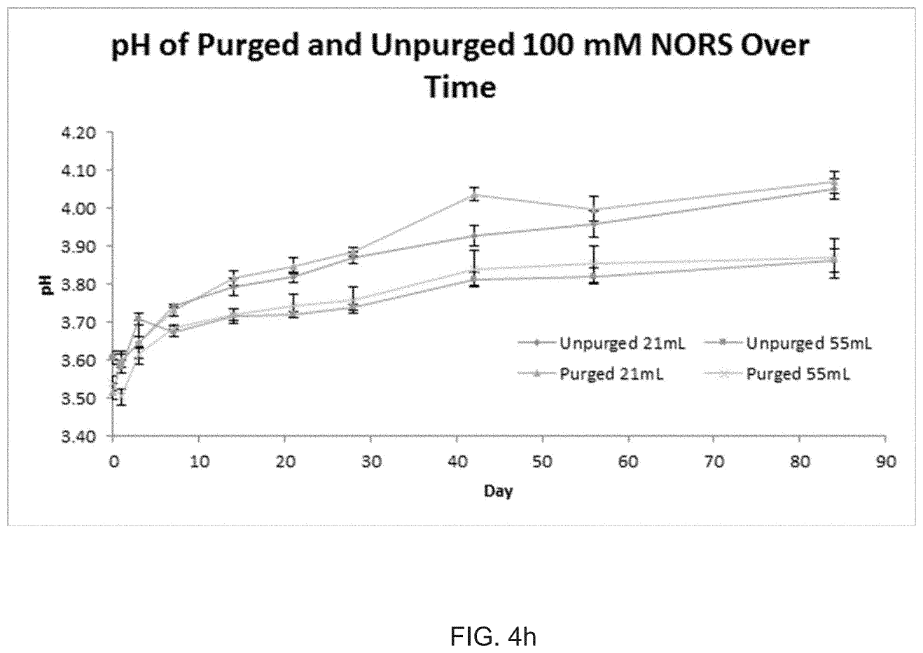

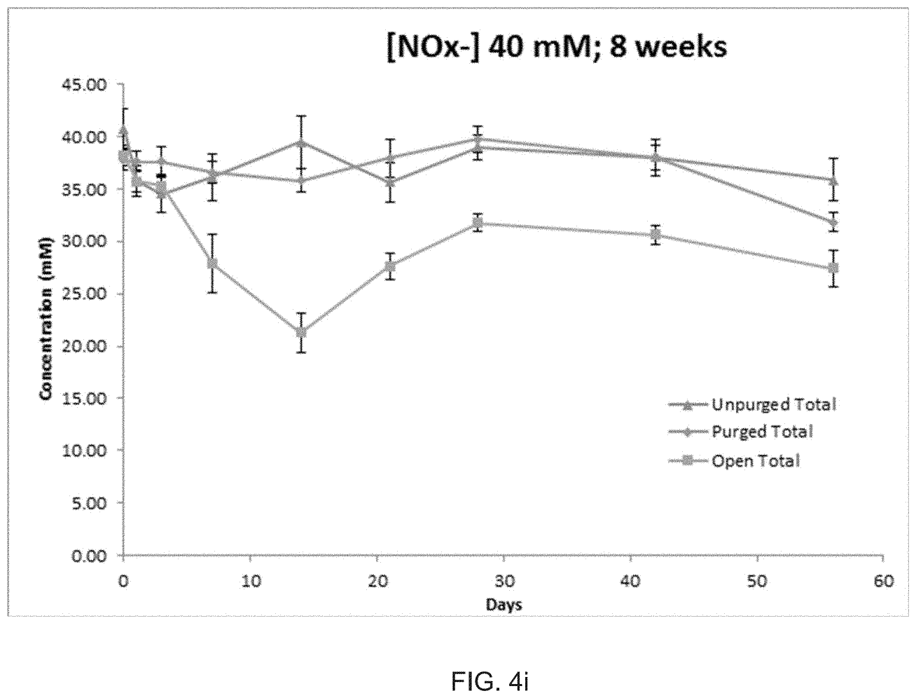

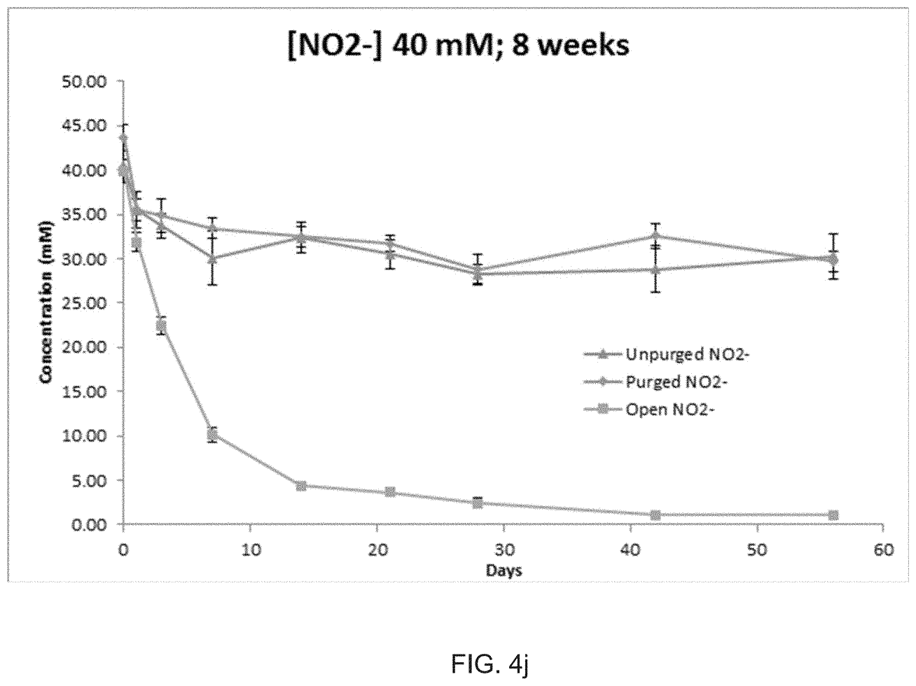

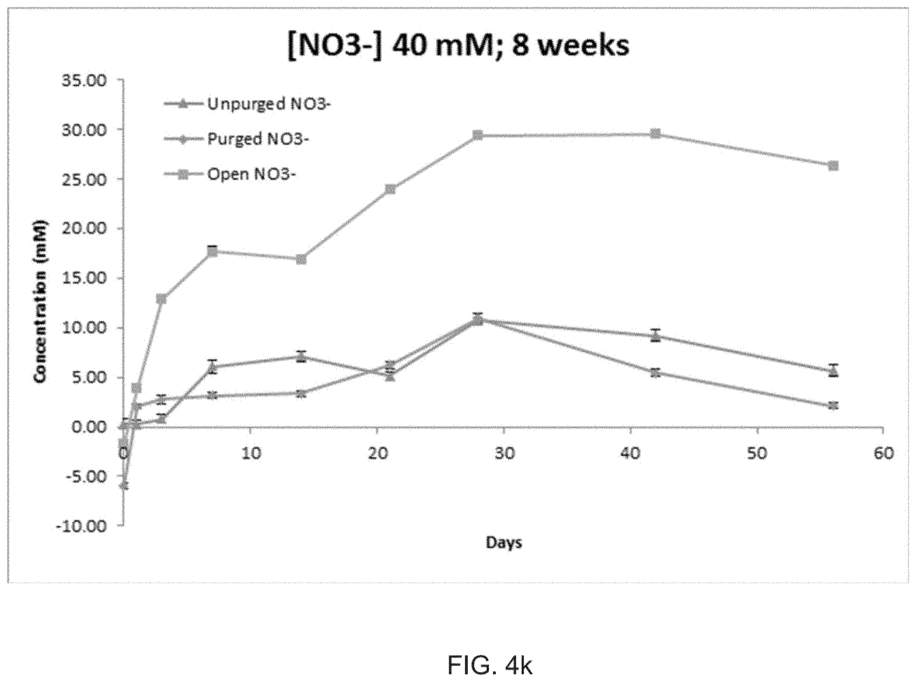

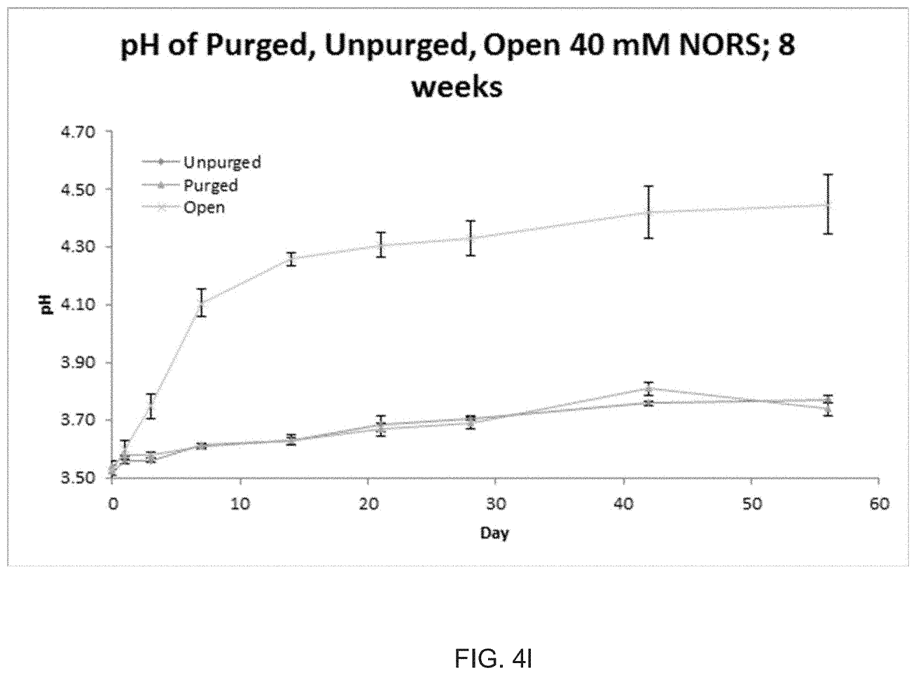

As depicted in FIG. 4a, over the first 24 hours the total nitrogen and nitrite concentrations appear stable though nitrite concentrations rise and then level off. Nitrite concentration showed reduction immediately after activation for both sizes of bottle, and it stayed stable until 48 hours when they started gradually declining. As depicted in FIG. 4b, total loss of nitrites over the 22 week period was 11.79+/−0.7 mM for the 21 mL samples, and 9.85+/−1.3 mM for the 55 mL samples, assuming the samples began with 20 mM NaNO2. This was a loss of about 60% and 50% for the smaller and larger bottle respectively.

Total nitrogen concentration in the samples remained stable for the first two weeks with some fluctuations. After those two weeks the total nitrogen concentration began to slowly decline. For the 21 mL samples there was a loss of 37% with a final concentration of 12.54+/−1.1 mM, and the 55 mL samples showed a loss of 34% with a final concentration of 13.14+/−0.5 mM total nitrogen after 22 weeks.

Though pH was not measured initially for these samples, FIG. 4c clearly shows that over time in the environment of this experiment the pH of NORS increases over long term storage. By week 3 the pH had only raised by 0.2 to 0.3 pH units; however, as time went on that pH continued to increase even until the last measurement 22 weeks after activation.

### 100 mM Unpurged and Purged

As depicted in FIG. 4d, after activation the unpurged 100 mM NORS started with a total nitrogen concentration of 95.89+/−3.1 mM in the 21 mL samples and 97.46+/−2.4 mM in the 55 mL samples; the nitrite concentration in the same order was 81.70+/−9.1 mM and 77.58+/−1.8 mM. As volumes were extracted for measuring there was a noticeable smell that coincides with the release of nitric oxide into the atmosphere, and periodically a small leak would have to be stopped with a gloved finger after the syringe was removed from the stopper. Pressure also appeared to have built up within the bottles as the stoppers bulged slightly and the syringe for pH sampling would fill itself after it was inserted into an inverted bottle. These observations remained constant over the first week which is also made evident by the relative stability of the NORS depicted in FIG. 4d.

Purged 100 mM NORS was measured on day zero to contain 92.84+/−2.1 mM and 99.33+/−2.0 mM total nitrogen and 93.41+/−4.7 mM and 94.20+/−2.0 mM nitrite concentration in 21 mL and 55 mL samples respectively. After the first week those concentrations had dropped to 93.04+/−2.7 mM and 96.70+/−2.2 mM total nitrogen and 84.95+/−3.5 mM and 88.45+/−2.9 mM nitrite respectively, as depicted in FIG. 4e. Similar observations of minor leaks, pressure build up, and smells of nitric oxide when measuring pH were noted during initial stages of the experiment.

Both unpurged and purged 100 mM NORS samples showed similar patterns when it came to stability. Both types showed relative stability up until the 1-month mark. The total concentration of nitrogen species remained in the 90 mM range, but the nitrite concentration drops in the initial few days and then stays stable for the first month. After week 4, unpurged total nitrogen and nitrites began to slowly decrease, as depicted in FIG. 4f, but purged nitrites didn't show they were decreasing until after week 6 when total nitrogen concentration showed reduction after week 4, as depicted in FIG. 4g. As for nitrate concentrations, they gradually rose until their high point on week 8, and then sharply decreased by week 12 after what appeared to be a loss of total nitrogen concentration but stability in the nitrite concentration. By the end of week 12 the total concentration of nitrogen species had dropped by 30 to 35%, but the nitrite concentration in both the unpurged and purged samples had dropped by 40 to 50%, as depicted in FIGS. 4f and 4g.

As depicted in FIG. 4h, the solution's pH was measured on the same day as concentration measurements were taken and recorded. Day 0 pH measurements were 3.61+/−0.01 and 3.60+/−0.01 for the unpurged 100 mM NORS and 3.52+/−0.02 and 3.54+/−0.02 for the purged 100 mM NORS in 21 mL and 55 mL samples respectively. The pH of the samples increased by about 0.13 and 0.08 in the unpurged 21 mL and 55 mL samples over the first week and in that same time the pH of the purged samples increased by 0.15 in the 55 mL samples and 0.21 in the 21 mL samples, as depicted in FIG. 4h. By week twelve the pH of the all the sample types was 4.07+/−0.03 in the purged 21 mL samples, 4.05+/−0.03 in the unpurged 21 mL samples, 3.87+/−0.05 in the purged 55 mL samples and 3.86+/−0.03 in the unpurged 55 mL samples.

### 40 mM Unpurged, Purged and Open System

Over the 8 weeks the 40 mM samples were tested there was an evident difference between the closed system and open system types of storage. Considering only nitrite concentrations for the unpurged, purged, and open system samples they each measured 40.46+/−1.7 mM, 43.65+/−1.5 mM, and 39.89+/−1.3 mM respectively. By the end of the 8 weeks those concentrations had dropped to 30.27+/−2.6 mM, 29.72+/−1.2 mM, and 1.08+/−0.4 mM. Both closed system samples dropped by close to 25% in their nitrite concentration, but the open system nitrite concentration decreased by close to 98%.

Both the purged and unpurged 40 mM NORS samples showed similar patterns of stability. Total nitrogen concentration stayed quite stable reducing to 38.97+/−1.2 mM in the unpurged sample and 39.74+/−1.3 mM in the purged samples; however, total nitrogen concentration dropped over the next two weeks to 35.88+/−2.0 and 31.82+/−0.9 respectively on week 6 when the nitrite concentration remained stable through that time as depicted in FIGS. 4i, 4j, and 4k. In contrast, the first week of the open sample tests showed a drop in their total nitrogen concentration from a measured 38.13+/−1.0 mM to 27.84+/−2.8 mM. After that week the total nitrogen concentration of the open system continued to measure close to that concentration up until the last measurements on week 8 where the concentration was 27.41+/−1.8 mM.

The pH of all three systems—purged and closed; unpurged and closed; and unpurged and open—started out very similar, as depicted in FIG. 4l. The three systems measured 3.54+/−0.01, 3.52+/−0.01, and 3.52+/−0.04 respectively, but the similarities of the open and closed systems ended there. By the end of the first week the open system samples had increased their pH to 4.11+/−0.05 when the closed system samples had only increased to 3.61+/−0.01 in that same time. This pattern continued over the 8 weeks to where the open system measured 4.45+/−0.1 and the closed purged and unpurged samples measured 3.74+/−0.02 and 3.77+/−0.01 respectively.

One unexpected result from this experiment was that the pH of the NORS stored in glass increased over time but the opposite has occurred with plastic storage options. Furthermore, opening the NORS to the atmosphere increased the rate at which the pH rose, and the rate remained constant until the supply of nitrite was considerably reduced at the end of the first week. Another unexpected result was that in the case of the closed 100 mM and 40 mM NORS there was a loss of nitrates after the first month but prior to that nitrites reduced at a slower rate or stayed constant.

The data indicates that the first month after activation was the most stable time for NORS after reaching an equilibrium in the crimp sealed bottles. The difference between the purged and unpurged solutions was relatively low. Differences were evident only in the first two days of the 100 mM tests, but after that time there was little difference between the behaviors of the two solutions. For the 40 mM tests there was little difference between the purged and unpurged solutions.

The swollen and soggy appearance of the rubber stoppers indicates possible reaction with the solutions or the gNO in the bottles headspace. A Teflon lined septum for each bottle can potentially reduce the probability of gNO reacting with its surroundings.

### Example 4—Determination of the Stability of NORS after Activation in an Open System

### Purpose:

To explore how stable NORS is in terms of nitric oxide production after activation with the tube uncapped for an extended period of time. NORS was injected into the flow-over device and measurements were gathered of the peak NO, nitric oxide production after 2 minutes, and the area under the curve after two minutes of 20 mM NORS uncapped and activated over 0, 10, 20, 30, 60, 120, 240 and 480 minutes.

### Methods:

The flow-over glass device was set-up as follows. The glass device was attached to a stand in a horizontal position. The inlet was connected to an inert gas (nitrogen) at a flow rate of 1 L/min. The exit was connected to a tube which was connected to the nitric oxide analyzer chemiluminescence.

20 mM NORS was prepared by mixing citric acid with a 20 mM sodium nitrite solution to reach a pH of 3.5. The NORS was uncapped for a period of time (0, 10, 20, 30, 60, 120, 240 or 480 minutes). Five milliliters of NORS was injected into the flow-over glass device and the data was collected using a bag.exe program for two minutes. Data was analyzed using GraphPad Prism 6. Each time point was repeated three times.

### Results:

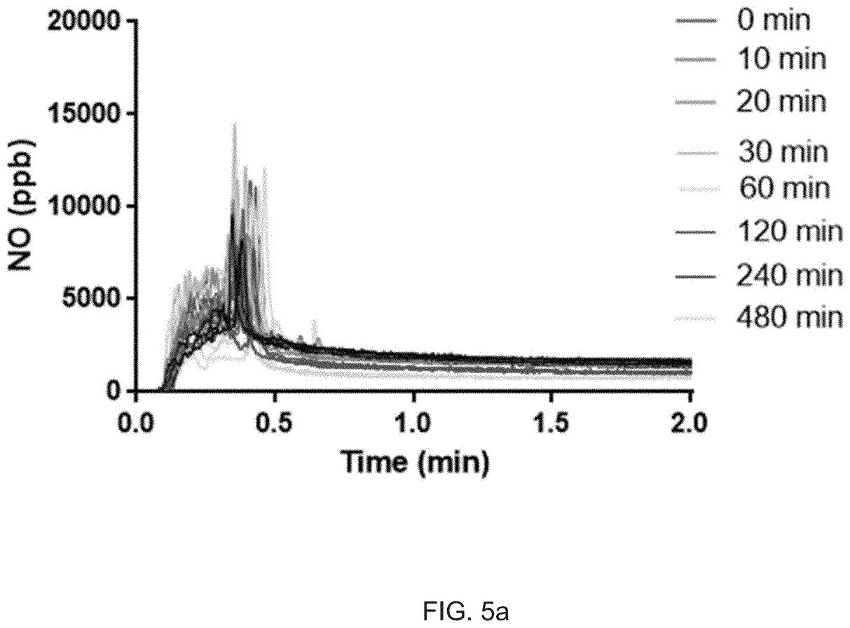

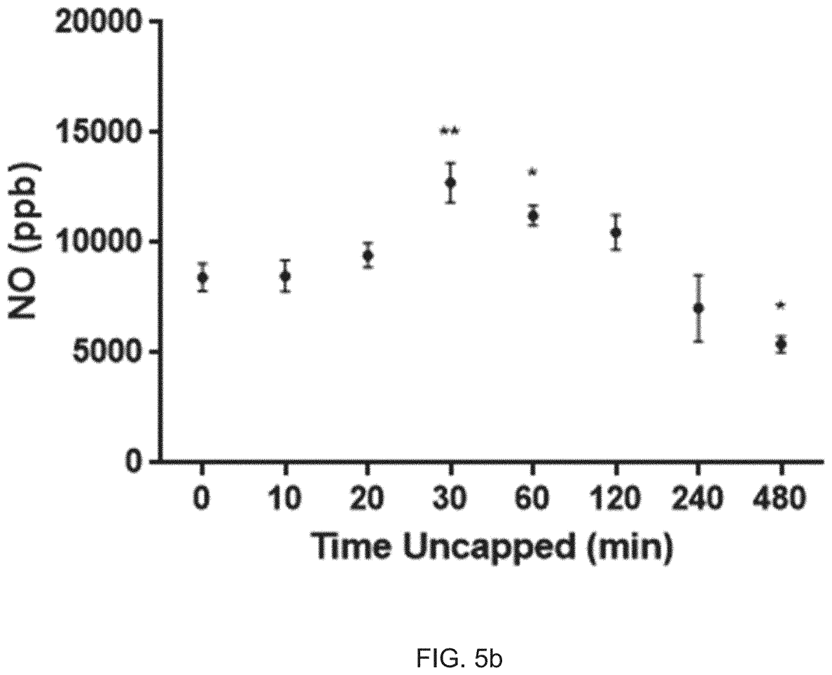

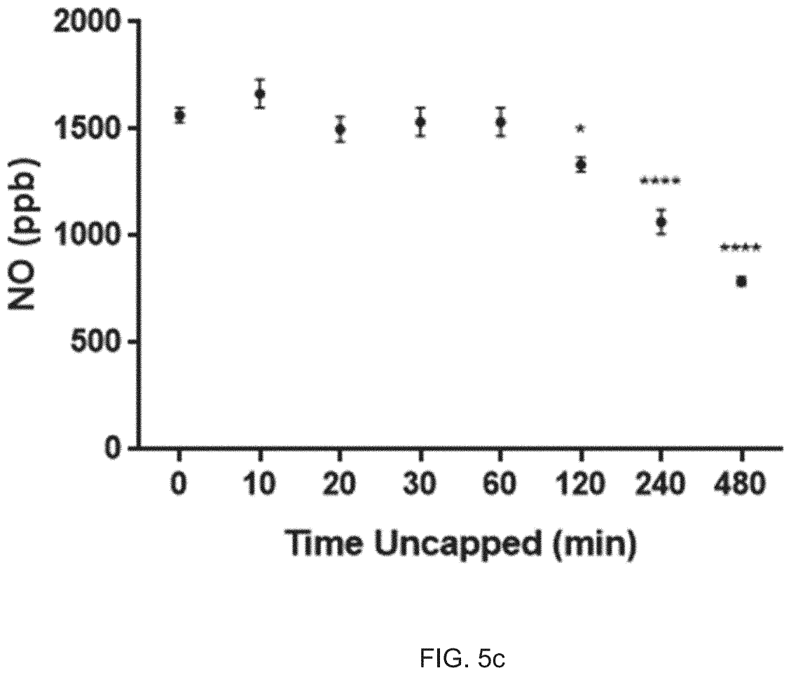

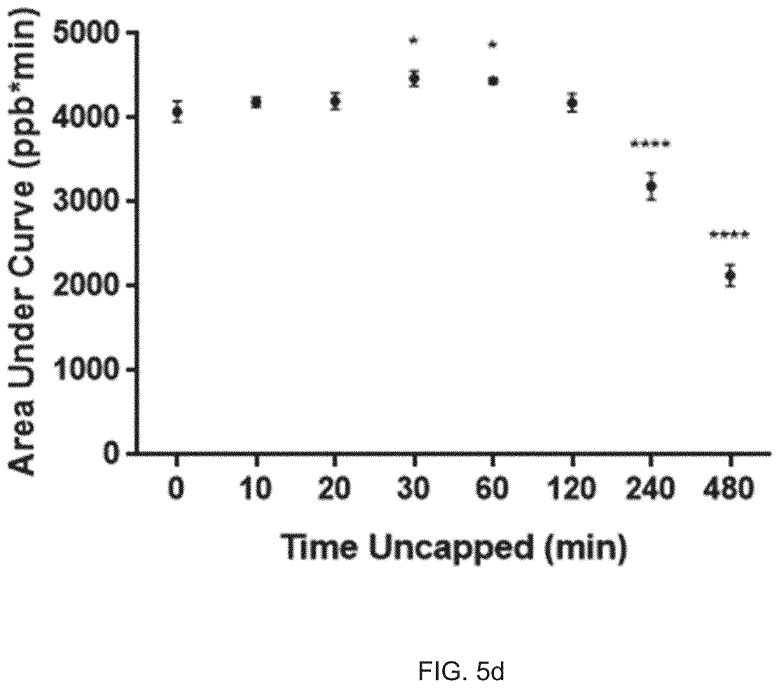

Curves obtained from chemiluminescence are depicted in FIG. 5a. Peak NO production was significantly higher relative to baseline (0 minutes uncapped) after the NORS was uncapped for 30 and 60 minutes and significantly lower after 480 minutes, as depicted in FIG. 5b. Nitric oxide production after two minutes was significantly lower relative to the baseline after the NORS was uncapped for 120, 240 and 480 minutes, as depicted in FIG. 5c. The area under the curve was significantly higher relative to the baseline after the NORS was uncapped for 30 minutes and significantly lower after 240 and 480 minutes, as depicted in FIG. 5d.

### Discussion:

Over time, the production of nitric oxide is reduced if the solution is uncapped for a period of 4 hours. Interestingly the 30 and 60 minute uncapped injections saw a larger peak and area under the curve relative to the baseline. Perhaps this is due to the differences in manually injecting the solution into the flow-over glass device. The added energy during injection would likely cause a larger peak and with only a two-minute measurement the injection time accounts for a large percentage of the area under the curve.

### Example 5—Stability Testing and Bacterial Growth

### Cohort #1 (Gel at Room Temperature)

The storage conditions for the samples was at room temperature of 20±2 degrees Celsius on a dark shelf in a 500 mL bottle. pH of the samples was measured with a pH meter. Nitrites were measured using Griess reagent and Chemiluminescence. Microbial contamination and microbial effect were determined. Results are depicted in Tables 3A and 3B.

TABLE 3A

| | | | | | | Bacterial |
|---|---|---|---|---|---|---|
| Date | Sample | Appearance | Nitrites | Nitrites | | growth |
| DD/MM/YYYY | # | Color | (Griess) | (Chemi) | pH | (Y/N) |
| Baseline | | | | | | |
| 28/11/2018 | 1 | Off-White | 64.9 | 61.8 | 3.56 | N |
| 28/11/2018 | 2 | Off-White | 73.9 | 68.8 | 3.57 | N |
| 28/11/2018 | 3 | Off-White | 73.5 | 69.9 | 3.56 | N |
| 28/11/2018 | 4 | Off-White | 78.4 | 74.8 | 3.51 | N |
| 28/11/2018 | 5 | Off-White | 73.2 | 69.5 | 3.57 | N |
| 1 Week | | | | | | |
| 05/12/2018 | 1 | Yellow-White | 71.2 | 65.8 | 3.60 | N |
| 05/12/2018 | 2 | Yellow-White | 71.9 | 66.7 | 3.61 | N |
| 05/12/2018 | 3 | Yellow-White | 70.9 | 66.3 | 3.60 | N |
| 05/12/2018 | 4 | Yellow-White | 70.9 | 64.1 | 3.58 | N |
| 05/12/2018 | 5 | Yellow-White | 71.5 | 66.4 | 3.60 | N |
| 2 Weeks | | | | | | |
| 12/12/2018 | 1 | Yellow-White | 61.5 | 64.4 | 3.63 | N |
| 12/12/2018 | 2 | Yellow-White | 65.6 | 67.3 | 3.62 | N |
| 12/12/2018 | 3 | Yellow-White | 65.3 | 65.9 | 3.57 | N |
| 12/12/2018 | 4 | Yellow-White | 73.2 | 76.1 | 3.62 | N |
| 12/12/2018 | 5 | Yellow-White | 69.7 | 72.0 | 3.63 | N |
| 1 Month | | | | | | |
| 03/01/2019 | 1 | Yellow-White | 54.2 | 51.7 | 3.66 | N |
| 03/01/2019 | 2 | Yellow-White | Malfunction | | 3.67 | N |
| 03/01/2019 | 3 | Yellow-White | 42.4 | 40.8 | 3.65 | N |
| 03/01/2019 | 4 | Yellow-White | 54.9 | 57.3 | 3.65 | N |
| 03/01/2019 | 5 | Yellow-White | 56.6 | 52.9 | 3.66 | N |

TABLE 3B

| Date | Sample | | | | |
|---|---|---|---|---|---|
| DD/MM/YYYY | # | Control | 30 sec | 60 sec | 90 sec |
| Baseline | | | | | |
| 28/11/2018 | 1 | 2.3*10⁵ | 8.4*10⁴ | 9.6*10³ | 1.3*10² |
| 28/11/2018 | 2 | | 7.9*10⁴ | 8.1*10³ | 3.1*10² |
| 28/11/2018 | 3 | | 6.3*10⁴ | 4.2*10³ | 9*10¹ |
| 28/11/2018 | 4 | | 9.7*10⁴ | 3.8*10³ | 1.8*10² |
| 28/11/2018 | 5 | | 7.3*10⁴ | 4.4*10³ | 2.1*10² |
| 1 Week | | | | | |
| 05/12/2018 | 1 | 2.4*10⁵ | 1.13*10⁵ | 2.45*10⁴ | 1.1*10³ |
| 05/12/2018 | 2 | | 1.26*10⁵ | 4.31*10⁴ | 1.7*10³ |
| 05/12/2018 | 3 | | 1.34*10⁵ | 2.32*10⁴ | 5.4*10³ |
| 05/12/2018 | 4 | | 8.0*10⁴ | 6.35*10⁴ | 1.9*10² |
| 05/12/2018 | 5 | | 1.69*10⁵ | 4.68*10⁴ | 3.2*10³ |
| 2 Weeks | | | | | |
| 12/12/2018 | 1 | 2.1*10⁵ | 4.8*10⁵ | 3 4*10⁴ | 1.38*10³ |
| 12/12/2018 | 2 | | 2.4*10⁵ | 2.7*10⁴ | 1.6*10² |
| 12/12/2018 | 3 | | 1.2*10⁵ | 5.2*10⁴ | 1.28*10³ |
| 12/12/2018 | 4 | | 5.0*10⁵ | 6.7*10⁴ | 4.4*10² |
| 12/12/2018 | 5 | | 3.5*10⁵ | 6.7*10⁴ | 2.7*10³ |
| 1 Month | | | | | |
| 03/01/2019 | 1 | 3.2*10⁵ | 3.3*10⁵ | 1.4*10⁵ | 1.5*10⁴ |
| 03/01/2019 | 2 | | 2.8*10⁵ | 1.9*10⁵ | 4.3*10⁴ |
| 03/01/2019 | 3 | | 3.2*10⁵ | 9*10⁴ | 7.3*10⁴ |
| 03/01/2019 | 4 | | 2.0*10⁵ | 1.0*10⁵ | 2.4*10⁴ |
| 03/01/2019 | 5 | | 2.1*10⁵ | 1.8*10⁵ | 2.9*10⁴ |

### Cohort #2 (Gel Accelerated)

The storage conditions for the samples was at a temperature of 40±2 degrees Celsius at a humidity of 65% in a dark incubator in a 500 mL bottle. pH of the samples was measured with a pH meter. Nitrites were measured using Griess reagent and Chemiluminescence. Microbial contamination and microbial effect were determined. Results are depicted in Tables 4A and 4B.

TABLE 4A

| | | | | | | Bacterial |
|---|---|---|---|---|---|---|
| Date | Sample | Appearance | Nitrites | Nitrites | | growth |
| DD/MM/YYYY | # | Color | (Griess) | (Chemi) | pH | (Y/N) |
| Baseline | | | | | | |
| 28/11/2018 | 1 | Off-White | 74.9 | 70.8 | 3.53 | N |
| 28/11/2018 | 2 | Off-White | 74.2 | 71.7 | 3.56 | N |
| 28/11/2018 | 3 | Off-White | 74.2 | 71.9 | 3.56 | N |
| 28/11/2018 | 4 | Off-White | 71.8 | 70.2 | 3.55 | N |
| 28/11/2018 | 5 | Off-White | 73.9 | 73.8 | 3.56 | N |
| 1 Week | | | | | | |
| 05/12/2018 | 1 | Off-White | 65.0 | 58.8 | 3.65 | N |
| 05/12/2018 | 2 | Off-White | 67.4 | 63.5 | 3.67 | N |
| 05/12/2018 | 3 | Off-White | 69.1 | 63.9 | 3.64 | N |
| 05/12/2018 | 4 | Off-White | 62.9 | 58.4 | 3.62 | N |
| 05/12/2018 | 5 | Off-White | 63.3 | 58.5 | 3.60 | N |
| 2 Weeks | | | | | | |
| 12/12/2018 | 1 | Off-White | 56.3 | 59.1 | 3.70 | N |
| 12/12/2018 | 2 | Off-White | 52.8 | 54.3 | 3.74 | N |
| 12/12/2018 | 3 | Off-White | 58.7 | 59.9 | 3.71 | N |
| 12/12/2018 | 4 | Off-White | 57.6 | 59.5 | 3.69 | N |
| 12/12/2018 | 5 | Off-White | 60.8 | 63.4 | 3.72 | N |
| 1 Month | | | | | | |
| 03/01/2019 | 1 | Off-White | 41.4 | 40.2 | 3.88 | N |
| 03/01/2019 | 2 | Off-White | 42.8 | 41.3 | 3.85 | N |
| 03/01/2019 | 3 | Off-White | 49.0 | 46.1 | 3.8 | N |
| 03/01/2019 | 4 | Off-White | 46.9 | 44.7 | 3.86 | N |
| 03/01/2019 | 5 | Off-White | 25.8 | 27.6 | 3.89 | N |

TABLE 4B

| Date | Sample | | | | |
|---|---|---|---|---|---|
| DD/MM/YYYY | # | Control | 30 sec | 60 sec | 90 sec |
| Baseline | | | | | |
| 28/11/2018 | 1 | 2.3*10⁵ | 7.6*10⁴ | 8.1*10³ | 8*10³ |
| 28/11/2018 | 2 | | 6.7*10⁴ | 4.7*10³ | 1.2*10² |
| 28/11/2018 | 3 | | 9.1*10⁴ | 7.4*10³ | 2.3*10² |
| 28/11/2018 | 4 | | 8.5*10⁴ | 9.6*10³ | 1.1*10² |
| 28/11/2018 | 5 | | 5.9*10⁴ | 7.8*10³ | 9*10¹ |
| 1 Week | | | | | |
| 05/12/2018 | 1 | 2.4*10⁵ | 1.67*10⁵ | 2.57*10⁴ | 2.31*10³ |
| 05/12/2018 | 2 | | 1.08*10⁵ | 1.72*10⁴ | 1.88*10³ |
| 05/12/2018 | 3 | | 8.7*10⁴ | 2.64*10⁴ | 4*10¹ |
| 05/12/2018 | 4 | | 1.33*10⁵ | 3.14*10⁴ | 1.52*10³ |
| 05/12/2018 | 5 | | 2.65*10⁵ | 6.73*10⁴ | 6.7*10² |
| 2 Weeks | | | | | |
| 12/12/2018 | 1 | 2.1*10⁵ | 3.0*10⁵ | 1.44*10⁵ | 4.40*10³ |
| 12/12/2018 | 2 | | 9.0*10⁵ | 1.57*10⁵ | 2.37*10³ |
| 12/12/2018 | 3 | | 3.0*10⁵ | 1.71*10⁵ | 1.19*10³ |
| 12/12/2018 | 4 | | 4.4*10⁵ | 8.7*10⁴ | 1.2*10² |
| 12/12/2018 | 5 | | 4.5*10⁵ | 4.5*10⁴ | 1.16*10³ |
| 1 Month | | | | | |
| 03/01/2019 | 1 | 3.2*10⁵ | 2.5*10⁵ | 2.3*10⁵ | 1.50*10⁵ |
| 03/01/2019 | 2 | | 2.7*10⁵ | 2.2*10⁵ | 1.19*10⁵ |
| 03/01/2019 | 3 | | 2.6*10⁵ | 5.7*10⁵ | 1.02*10⁵ |
| 03/01/2019 | 4 | | 6.4*10⁵ | 3.5*10⁵ | 1.77*10⁵ |
| 03/01/2019 | 5 | | 3.2*10⁵ | 5.0*10⁵ | 1.49*10⁵ |

### Cohort #3 (Liquid at Room Temperature)

The storage conditions for the samples was at a temperature of 20±2 degrees Celsius on a dark shelf in a 500 mL bottle. pH of the samples was measured with a pH meter. Nitrites were measured using Griess reagent and Chemiluminescence. Microbial contamination and microbial effect were determined. Results are depicted in Tables 5A and 5B.

TABLE 5A

| | | | | | | Bacterial |
|---|---|---|---|---|---|---|
| Date | Sample | Appearance | Nitrites | Nitrites | | growth |
| DD/MM/YYYY | # | Color | (Griess) | (Chemi) | pH | (Y/N) |
| Baseline | | | | | | |
| 13/12/2018 | 1 | Clear | 15.0 | 15.4 | 3.54 | N |
| 13/12/2018 | 2 | Clear | 15.4 | 15.5 | 3.54 | N |
| 13/12/2018 | 3 | Clear | 14.8 | 14.6 | 3.51 | N |
| 13/12/2018 | 4 | Clear | 15.3 | 15.7 | 3.51 | N |
| 13/12/2018 | 5 | Clear | 14.9 | 16.1 | 3.55 | N |
| 1 Week | | | | | | |
| 20/12/2018 | 1 | Clear | 13.5 | 13.2 | 3.55 | N |
| 20/12/2018 | 2 | Clear | 13.9 | 13.1 | 3.55 | N |
| 20/12/2018 | 3 | Clear | 13.0 | 12.6 | 3.51 | N |
| 20/12/2018 | 4 | Clear | 13.2 | 12.9 | 3.53 | N |
| 20/12/2018 | 5 | Clear | 13.7 | 13.6 | 3.55 | N |
| 1 Month | | | | | | |
| 10/01/2019 | 1 | Clear | 10.7 | 11.0 | 3.41 | N |
| 10/01/2019 | 2 | Clear | 10.7 | 11.3 | 3.45 | N |
| 10/01/2019 | 3 | Clear | 9.8 | 10.8 | 3.39 | N |
| 10/01/2019 | 4 | Clear | 10.3 | 10.9 | 3.41 | N |
| 10/01/2019 | 5 | Clear | 10.5 | 11.3 | 3.43 | N |

TABLE 5B

| Date | Sample | | | | |
|---|---|---|---|---|---|
| DD/MM/YYYY | # | Control | 2 min | 5 min | 10 min |
| Baseline | | | | | |
| 13/12/2018 | 1 | 1.1*10⁵ | 9*10⁴ | 5.1*10⁴ | 2.7*10³ |
| 13/12/2018 | 2 | | 1.0*10⁵ | 3.0*10⁴ | 2.2*10³ |
| 13/12/2018 | 3 | | 1.6*10⁵ | 2.9*10⁴ | 1.5*10³ |
| 13/12/2018 | 4 | | 1.3*10⁵ | 4.2*10⁴ | 3.7*10³ |
| 13/12/2018 | 5 | | 7*10⁴ | 3.0*10⁴ | 1.2*10³ |
| 1 Week | | | | | |
| 20/12/2018 | 1 | 3.0*10⁵ | 4.1*10⁵ | 1.05*10⁵ | 1.5*10⁴ |
| 20/12/2018 | 2 | | 2.6*10⁵ | 1.12*10⁵ | 2.7*10⁴ |
| 20/12/2018 | 3 | | 3.2*10⁵ | 1.35*10⁵ | 2.0*10⁴ |
| 20/12/2018 | 4 | | 2.0*10⁵ | 1.30*10⁵ | 1.1*10⁴ |
| 20/12/2018 | 5 | | 2.1*10⁵ | 9.8*10⁴ | 1.0*10⁴ |
| 1 Month | | | | | |
| 10/01/2019 | 1 | 1.9*10⁵ | 2.3*10⁵ | 1.4*10⁵ | 5.9*10⁴ |
| 10/01/2019 | 2 | | 2.6*10⁵ | 1.8*10⁵ | 3.2*10⁴ |
| 10/01/2019 | 3 | | 2.6*10⁵ | 2.0*10⁵ | 6.5*10⁴ |
| 10/01/2019 | 4 | | 1.9*10⁵ | 2.5*10⁵ | 6.7*10⁴ |
| 10/01/2019 | 5 | | 2.0*10⁵ | 9*10⁴ | 2.8*10⁴ |

### Cohort #4 (Liquid Accelerated)

The storage conditions for the samples was at a temperature of 40±2 degrees Celsius at a humidity of 65% in a dark incubator in a 500 mL bottle. pH of the samples was measured with a pH meter. Nitrites were measured using Griess reagent and Chemiluminescence. Microbial contamination and microbial effect were determined. Results are depicted in Tables 6A and 6B.

TABLE 6A

| | | | | | | Bacterial |
|---|---|---|---|---|---|---|
| Date | Sample | Appearance | Nitrites | Nitrites | | growth |
| DD/MM/YYYY | # | Color | (Griess) | (Chemi) | pH | (Y/N) |
| Baseline | | | | | | |
| 13/12/2018 | 1 | Clear | 15.5 | 15.4 | 3.51 | N |
| 13/12/2018 | 2 | Clear | 15.9 | 15.9 | 3.51 | N |
| 13/12/2018 | 3 | Clear | 15.4 | 15.2 | 3.53 | N |
| 13/12/2018 | 4 | Clear | 15.7 | 16.2 | 3.52 | N |
| 13/12/2018 | 5 | Clear | 15.6 | 15.7 | 3.50 | N |
| 1 Week | | | | | | |
| 20/12/2018 | 1 | Clear | 10.6 | 10.2 | 3.53 | N |
| 20/12/2018 | 2 | Clear | 11.0 | 10.4 | 3.57 | N |
| 20/12/2018 | 3 | Clear | 11.6 | 10.8 | 3.58 | N |
| 20/12/2018 | 4 | Clear | 10.8 | 10.4 | 3.56 | N |
| 20/12/2018 | 5 | Clear | 10.5 | 9.6 | 3.54 | N |
| 1 Month | | | | | | |
| 10/01/2019 | 1 | Clear | 1.7 | 3.2 | 3.22 | N |
| 10/01/2019 | 2 | Clear | 2.1 | 3.3 | 3.24 | N |
| 10/01/2019 | 3 | Clear | 2.1 | 3.1 | 3.25 | N |
| 10/01/2019 | 4 | Clear | 1.9 | 3.1 | 3.27 | N |
| 10/01/2019 | 5 | Clear | 1.6 | 2.8 | 3.17 | N |

TABLE 6B

| Date | Sample | | | | |
|---|---|---|---|---|---|
| DD/MM/YYYY | # | Control | 2 min | 5 min | 10 min |
| Baseline | | | | | |
| 13/12/2018 | 1 | 1.1*10⁵ | 1.5*10⁵ | 3.9*10⁴ | 2.7*10³ |
| 13/12/2018 | 2 | | 1.0*10⁵ | 3.7*10⁴ | 2.1*10³ |
| 13/12/2018 | 3 | | 6*10⁴ | 3.4*10⁴ | 1.6*10³ |
| 13/12/2018 | 4 | | 9*10⁴ | 2.1*10⁴ | 1.8*10³ |
| 13/12/2018 | 5 | | 6*10⁴ | 2.7*10⁴ | 1.7*10³ |
| 1 Week | | | | | |
| 20/12/2018 | 1 | 3.0*10⁵ | 2.7*10⁵ | 1.48*10⁵ | 2.8*10⁴ |
| 20/12/2018 | 2 | | 1.5*10⁵ | 1.52*10⁵ | 4.7*10⁴ |
| 20/12/2018 | 3 | | 4.1*10⁵ | 1.59*10⁵ | 3.2*10⁴ |
| 20/12/2018 | 4 | | 3.8*10⁵ | 1.70*10⁵ | 5.4*10⁴ |
| 20/12/2018 | 5 | | 2.0*10⁵ | 1.61*10⁵ | 3.2*10⁴ |
| 1 Month | | | | | |
| 10/01/2019 | 1 | 1.9*10⁵ | 2.2*10⁵ | 1.9*10⁵ | 1.66*10⁵ |
| 10/01/2019 | 2 | | 2.8*10⁵ | 2.3*10⁵ | 1.82*10⁵ |
| 10/01/2019 | 3 | | 2.6*10⁵ | 2.1*10⁵ | 1.40*10⁵ |
| 10/01/2019 | 4 | | 2.7*10⁵ | 2.0*10⁵ | 1.53*10⁵ |
| 10/01/2019 | 5 | | 3.3*10⁵ | 1.9*10⁵ | 1.67*10⁵ |

It should be understood that the above-described methods are only illustrative of some embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, may be made without departing from the principles and concepts set forth herein.

What is claimed is:

1. A gas-evolving composition delivery system, comprising:
a pressurized container having a dispenser; and
a liquid gas-evolving composition within the pressurized container, said gas-evolving composition, comprising:
a gas-evolving donor,
an activator, and
a pharmaceutically acceptable carrier,
wherein the pressurized container has an internal pressure sufficient to minimize gas production of the gas-evolving composition while within the container.

2. The system of claim 1, further comprising a propellant within the pressurized container.

3. The system of claim 1, wherein the pressurized container is a bag-on-valve container, a bag-in-bag container, a piston can container, or a can-in-can container.

4. The system of claim 1, wherein the pressurized container is a respiratory inhaler or an aerosol container.

5. The system of claim 1, wherein an internal pressure of the pressurized container is from 150 kilopascals (kPa) to 1000 kPa.

6. The system of claim 1, wherein the dispensing actuator is a metered dose dispensing actuator.

7. The system of claim 1, wherein the dispensing actuator is a continuous flow dispensing actuator.

8. The system of claim 2, wherein the propellant and the gas-evolving composition are physically separated.

9. The system of claim 2, wherein the propellant and the gas-evolving composition are combined.

10. The system of claim 2, wherein the propellant comprises a volatile hydrocarbon, dimethyl ether, methyl ethyl ether, air, nitrogen, nitrous oxide, helium, argon, carbon dioxide, a hydrofluoroalkane (HFA), or a combination thereof.

11. The system of claim 1, wherein the gas-evolving composition has a viscosity of greater than or equal to 5000 centipoise (cps).

12. The system of claim 1, wherein the gas-evolving composition is formulated to evolve carbon dioxide gas.

13. The system of claim 12, wherein the gas-evolving donor comprises carbonic acid, bicarbonate, carbonate, a salt thereof, a complex thereof, or a combination thereof.

14. The system of claim 12, wherein the activator comprises a hydroxy acid, citric acid, ascorbic acid, lactic acid, glycolic acid, salicylic acid, or a combination thereof.

15. The system of claim 12, wherein the pH of the gas-evolving composition is from about 1 to about 7.

16. The system of claim 1, wherein the gas-evolving composition is formulated to evolve oxygen gas.

17. The system of claim 16, wherein the gas-evolving donor comprises hydrogen peroxide, a superoxide, or a combination thereof.

18. The system of claim 16, wherein the activator comprises a catalase, a peroxidase, manganese dioxide, iron, or a combination thereof.

19. The system of claim 1, wherein the gas-evolving composition is formulated to evolve nitric oxide gas.

20. The system of claim 19, wherein the gas-evolving donor comprises a nitrite or nitrite salt, a nitrate or nitrate salt, sodium nitroprusside, molsidomine or a metabolite thereof, a diazeniumdiolates, a S-nitrosothiol, mesoionic oxatriazole, iron-sulphur nitrosyls, sinitrodil, a NONOate compound, or a combination thereof.

21. The system of claim 19, wherein the activator comprises a metal, a hydroxy acid, citric acid, ascorbic acid, lactic acid, glycolic acid, salicylic acid, hydrochloric acid, nitric acid, sulfuric acid, phosphoric acid, sodium iodide, iron sulfate, potassium nitrate, a chromate salt, or a combination thereof.

22. The system of claim 19, wherein the gas-evolving composition has a pH of from about 1 to about 7.

23. The system of claim 1, wherein the gas-evolving composition is formulated to evolve carbon monoxide.

24. The system of claim 23, wherein the gas-evolving donor comprises a transition metal carbon monoxide releasing molecule (CORM), a photoCORM, an enzyme-triggered CORM, an organic CORM, an enzyme hybrid CORM (HYCO), a carbon monoxide releasing material (CORMA), carboxymethylhemoglobin, a porphyrin, or a combination thereof.

25. The system of claim 1, wherein the gas-evolving composition further comprises an additional active agent.

26. The system of claim 25, wherein the additional active agent comprises a steroid, a sebostatic agent, a keratolytic agent, an herbal extract, an acaricide, an age spot or keratose removing agent, an allergen, an analgesic, an antiacne agent, an antiallergic agent, an antiaging agent, an anti-bacterial, an antibiotic, an antiburn agent, an anticancer agent, an antidandruff agent, an antidepressant, an anti-dermatitis agent, an anti-edemic agent, an antihistamine, an anti-helminth, an anti-hyperkeratolyte agent, an anti-inflammatory agent, an anti-irritant, an anti-lipemic, an antimicrobial, an antimycotic, an antiproliferative agent, an antioxidant, an anti-wrinkle agent, an anti-pruritic, an anti-psoriatic agent, an anti-rosacea agents anti-seborrheic agents, antiseptic, anti-swelling agents, antiviral agents, anti-yeast agent, an astringent, a topical cardiovascular agent, a chemotherapeutic agent, a corticosteroid, a dicarboxylic acid, a disinfectant, a fungicide, a hair growth regulator, a hormone, a hydroxy acid, an immunosuppressant, an immunoregulating agent, an insecticide, an insect repellent, a keratolytic agent, a lactam, a metal, a local anesthetic, a metal oxides, a mitocide, a neuropeptides, a non-steroidal anti-inflammatory agent, an oxidizing agent, a pediculicides, a photodynamic therapy agent, a retinoid, a scabicide, a self-tanning agent, a skin whitening agent, a vasoconstrictor, a vasodilator, a vitamin, a vitamin D derivative, a wound healing agent, a wart remover, or a combination thereof.

27. The system of claim 1, wherein the pharmaceutically acceptable carrier is an aqueous liquid.

28. The system of claim 1, wherein the pharmaceutically acceptable carrier is a hydrophobic carrier.

29. The system of claim 1, wherein the gas-evolving composition further comprises a gelling agent, a polymeric agent, an emollient, a polar solvent, a surface-active agent, an oil, or a combination thereof.

30. The system of claim 1, wherein the gas-evolving composition is formulated as a liquid, a gel, or an emulsion.

31. The system of claim 1, wherein an amount of oxygen in a head space of the container is less than a predefined threshold.

32. A method of treating a disease or condition of a subject, comprising:
administering a gas-evolving composition to the subject from a pressurized container, wherein an internal pressure of the pressurized container minimizes gas production of the gas-evolving composition prior to administration.

33. The method of claim 32, wherein the disease or condition comprises a dermatological disorder, pain, infection, a hormonal disorder, a cardiovascular disorder, a respiratory disorder, gastrointestinal disorder, a gynecological disorder, a sexually-transmitted disease, a wound, or a combination thereof.

34. The method of claim 32, wherein the gas-evolving composition is administered to one or more of the skin, a mucosal membrane, the anus, the rectum, the GI system, the vagina, the penile urethra, an eye, the respiratory system, the oral cavity, the nasal cavity, the sinuses, the pharynx, the larynx, the trachea, the bronchus, the lungs, the dental system, and the ear canal.

35. The method of claim 32, wherein the gas-evolving composition is administered to the subject as an aerosol, a foam, a gel, or an emulsion.

36. The method of claim 32, wherein administering the gas-evolving composition further comprises administering carbon dioxide gas, oxygen gas, nitric oxide gas, carbon monoxide gas, or a combination thereof to the subject in an amount sufficient to treat the disease or disorder.

37. The method of claim 32, wherein administering further comprises administering an additional therapeutic agent.

38. The method of claim 37, wherein the additional therapeutic agent comprises a steroid, a sebostatic agent, a keratolytic agent, an herbal extract, an acaricide, an age spot or keratose removing agent, an allergen, an analgesic, an antiacne agent, an antiallergic agent, an antiaging agent, an anti-bacterial, an antibiotic, an antiburn agent, an anticancer agent, an antidandruff agent, an antidepressant, an anti-dermatitis agent, an anti-edemic agent, an antihistamine, an anti-helminth, an anti-hyperkeratolyte agent, an anti-inflammatory agent, an anti-irritant, an anti-lipemic, an antimicrobial, an antimycotic, an antiproliferative agent, an antioxidant, an anti-wrinkle agent, an anti-pruritic, an anti-psoriatic agent, an anti-rosacea agents anti-seborrheic agents, antiseptic, anti-swelling agents, antiviral agents, anti-yeast agent, an astringent, a topical cardiovascular agent, a chemotherapeutic agent, a corticosteroid, a dicarboxylic acid, a disinfectant, a fungicide, a hair growth regulator, a hormone, a hydroxy acid, an immunosuppressant, an immunoregulating agent, an insecticide, an insect repellent, a keratolytic agent, a lactam, a metal, a local anesthetic, a metal oxides, a mitocide, a neuropeptides, a non-steroidal anti-inflammatory agent, an oxidizing to agent, a pediculicides, a photodynamic therapy agent, a retinoid, a scabicide, a self-tanning agent, a skin whitening agent, a vasoconstrictor, a vasodilator, a vitamin, a vitamin D derivative, a wound healing agent, a wart remover, or a combination thereof.

39. A method of controlling gas release from a gas-evolving composition, comprising:
subjecting a gas-evolving composition to a release-controlling pressure within a container to minimize gas release from the gas-evolving composition while in the container, the gas-evolving composition comprising:
a gas-evolving donor,
an activator, and
a pharmaceutically acceptable carrier.

40. The method of claim 39, wherein the release-controlling pressure is from 150 kPa to 1000 kPa.

41. The method of claim 39, wherein the release-controlling pressure is from 300 kPa to 700 kPa.

42. The method of claim 39, wherein the release-controlling pressure is from 400 kPa to 800 kPa.

43. The method of claim 39, wherein the release-controlling pressure is from 500 kPa to 900 kPa.

44. The method claim 39, wherein the container is a bag-on-valve container, a bag-in-bag container, a piston can container, a can-in-can container, a respiratory inhaler, or an aerosol container.

45. The method of claim 39, wherein the gas-evolving composition is formulated to evolve carbon dioxide gas, nitric oxide gas, oxygen gas, carbon monoxide gas, or a combination thereof.

46. The method of claim 39, wherein the pharmaceutically acceptable carrier is an aqueous liquid.

47. The method of claim 39, wherein the pharmaceutically acceptable carrier is a hydrophobic liquid.

48. A method of manufacturing a gas-releasing system, comprising:
loading a gas-evolving composition into a container, the gas-evolving composition comprising:
a gas-evolving donor,
an activator, and
a pharmaceutically acceptable carrier; and
pressurizing the container to an internal pressure sufficient to minimize gas production of the gas-evolving composition while within the container using a propellant.

49. The method of claim 48, further comprising:
pressurizing the container to the internal pressure sufficient to minimize gas production of the gas-evolving composition while within the container using a propellant.

50. The method claim 48, wherein the container is a bag-on-valve container, a bag-in-bag container, a piston can container, a can-in-can container, a respiratory inhaler, or an aerosol container.

51. The method of claim 48, wherein the gas-evolving composition is formulated to evolve carbon dioxide gas, nitric oxide gas, oxygen gas, carbon monoxide gas, or a combination thereof.

52. The method of claim 48, wherein the pharmaceutically acceptable carrier is an aqueous liquid.

53. The method of claim 48, wherein the pharmaceutically acceptable carrier is a hydrophobic liquid.

54. The method of claim 48, wherein the internal pressure is from 150 kPa to 1000 kPa.

55. The method of claim 49, wherein the propellant and the gas-evolving composition are physically separated.

56. The method of claim 49, wherein the propellant and the gas-evolving composition are combined.

57. The method of claim 49, wherein the propellant comprises a volatile hydrocarbon, dimethyl ether, methyl ethyl ether, air, nitrogen, nitrous oxide, helium, argon, carbon dioxide, a hydrofluoroalkane (HFA), or a combination thereof.

58. A method of stabilizing an acidified nitrite solution, comprising:
loading the acidified nitrite solution into a container, the acidified nitrite solution comprising:
a nitric oxide (NO) evolving donor;
an NO activator;
a pharmaceutically acceptable carrier; and
sealing the container to be airtight.

59. The method of claim 58, further comprising reducing a headspace in the container to below a predetermined threshold amount.

60. The method of claim 58, further comprising reducing an amount of oxygen in a headspace of the container below a predetermined threshold.

61. The method of claim 58, wherein the acidified nitrite solution is configured to maintain minimum threshold donor and activator concentrations over a predetermined period of time.

62. The method of claim 61, wherein the threshold concentrations are at least one of:
a minimum threshold donor concentration of 2 millimolar (mM), 10 mM, 50 mM, 100 mM, 200 mM, or 500 mM; or
a minimum threshold activator concentration with a pH of 2.8-5.5.

63. The method of claim 61, wherein the period of time is at least one of: 7 days, 14 days, 30 days, or 60 days.

64. The method of claim 59, wherein the threshold amount of headspace is less than: 5% of the container, 4% of the container, 3% of the container, 2% of the container, 1% of the container, 0.5% of the container, 0.1% of the container, or 0.01% of the container.

65. The method of claim 60, wherein the threshold amount of oxygen in the head space of the container is less than: 21%, 5%, 2%, 1%, or 0.1%.

66. The method of claim 58, wherein the NO-evolving donor comprises a nitrite or nitrite salt, a nitrate or nitrate salt, sodium nitroprusside, molsidomine or a metabolite thereof, a diazeniumdiolates, a S-nitrosothiol, mesoionic oxatriazole, iron-sulphur nitrosyls, sinitrodil, a NONOate compound, or a combination thereof.

67. The method of claim 58, wherein the NO activator comprises a metal, a hydroxy acid, citric acid, ascorbic acid, lactic acid, glycolic acid, salicylic acid, hydrochloric acid, nitric acid, sulfuric acid, phosphoric acid, sodium iodide, iron sulfate, potassium nitrate, a chromate salt, or a combination thereof.

68. The method of claim 58, wherein the pharmaceutically acceptable carrier is at least one of an aqueous liquid or a hydrophobic carrier.

69. The method of claim 58, wherein the acidified nitrite solution further comprises a gelling agent, a polymeric agent, an emollient, a polar solvent, a surface-active agent, an oil, or a combination thereof.

70. The method of claim 58, wherein the acidified nitrite solution has a pH of from about 1 to about 7.
